SEQUENCE LISTING (1 sequence)

SEQ ID NO: 1 - protein, 443 aa, Homo sapiens
MECLRSLPCLLPRAMRLPRRTLCALALDVTSVGPPVAACGRRANLIGRSRAAQLCGPDRLRVAGEVHRFRTSDVSQATLASVAPVFTVTKFDKQGNVTSFERKKTELYQELGLQARDLRFQHVMSITVRNNRIIMRMEYLKAVITPECLLILDYRNLNLEQWLFRELPSQLSGEGQLVTYPLPFEFRAIEALLQYWINTLQGKLSILQPLILETLDALVDPKHSSVDRSKLHILLQNGKSLSELETDIKIFKESILEILDEEELLEELCVSKWSDPQVFEKSSAGIDHAEEMELLLENYYRLADDLSNAARELRVLIDDSQSIIFINLDSHRNVMMRLNLQLTMGTFSLSLFGLMGVAFGMNLESSLEEDHRIFWLITGIMFMGSGLIWRRLLSFLGRQLEAPLPPMMASLPKKTLLADRSMELKNSLRLDGLGSGRSILTNR

(19) United States
(12) Patent Application Publication
(10) Pub. No.: US 2024/0415878 A1
(43) Pub. Date: Dec. 19, 2024

(54) Compositions and Methods for Modulating Mitochondrial Function

(71) Applicant: Board of Regents, The University of Texas System, Austin, TX (US)

(72) Inventor: Madesh Muniswamy, San Antonio, TX (US)

(73) Assignee: Board of Regents, The University of Texas System, Austin, TX (US)

(21) Appl. No.: 18/701,303

(22) PCT Filed: Oct. 13, 2022
(86) PCT No.: PCT/US2022/046631

§ 371 Date: Apr. 15, 2024

Related U.S. Application Data
(60) Provisional application No. 63/255,077, filed on Oct. 13, 2021.

(51) Int. Cl. A61K 33/24; A61P 3/04

(52) U.S. Cl. CPC A61K 33/24; A61P 3/04

(57) ABSTRACT

The inventor has discovered that limiting mitochondrial Mg²⁺ uptake (ₘMg²⁺) prevents diet-induced obesity. Administration of compositions that limit mitochondrial Mg²⁺ uptake (ₘMg²⁺) can be used for various therapeutic and prophylactic uses.

### PRIORITY PARAGRAPH

This Application is an International Application claiming priority to U.S. Provisional Application Ser. No. 63/255,077 filed Oct. 13, 2021, which is incorporated herein by reference in its entirety.

### STATEMENT REGARDING FEDERALLY FUNDED RESEARCH

This invention was made with government support under R01GM109882 and R01HL086699 awarded by the National Institutes of Health (NIH). The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

The hallmarks of diet and aging associated metabolic syndrome are mitochondrial dysfunction, dysregulation of cellular ion homeostasis, metabolic perturbation, and inadequate nutrient and oxygen supply by microvascular density loss. Emerging evidence highlights the vital role of ion channels as regulators of mitochondrial bioenergetics and cellular metabolism including oxidative phosphorylation (OXPHOS), ATP production, mitochondrial integrity, mitochondrial volume, enzyme activity, signal transduction, proliferation, and apoptosis. Although the progressive changes were either correlated phenomena or circumstantial evidence, the direct cause and effect relationship has not been established. Substantial progress in developing small molecule medicines for metabolic and cardiovascular diseases is becoming increasingly apparent, the limited understanding of energy biology and the interrelationship between bioenergetics and diet influences are therefore obligatory. In multicellular organisms, mitochondrial Ca²⁺ uptake is fundamentally essential for cellular bioenergetics despite its influence on Ca²⁺-induced mitochondrial permeability pore activation leading to energetic collapse. Given that mitochondrial dysfunction involves a perturbation of metabolism and intracellular ion homeostasis, the direct link between the mitochondrial divalent ions flux and its buffering capacity that controls bioenergetics needs to be understood.

The impaired divalent cation dynamics at the cellular level is linked to mitochondrial dysfunction. Although mitochondria are the cellular hub for metabolism, any metabolic anomalies have been linked to several metabolic disorders including obesity, diabetes, and cardiovascular disease. The major mitochondrial Ca²⁺ uptake machinery MCU is essential to shape cytosolic Ca²⁺ dynamics and promote glucose and fatty acid oxidation dependent mitochondrial respiration. Genetic ablation of MCU in liver, cardiac, and skeletal tissues promote triglyceride accumulation, lowers ketone body production, and increases total body fat. The hepatic and extrahepatic lipid accumulation phenotype observed in the murine and zebrafish model systems suggest a conservation of MCU-regulated cellular metabolism across species. In contrast, several pieces of evidence link intracellular Mg²⁺ (ᵢMg²⁺) is an endogenous inactivator of several channels including mitochondrial Ca²⁺ uniporter channel that is essential to ignite TCA cycle for reducing equivalents production and ATP synthesis. Unlike mitochondrial Ca²⁺ dynamics, ₘMg²⁺ uptake machinery Mrs2 is conserved in both anaerobic and aerobic species. ᵢMg²⁺ is essential for wide range of cellular processes including membrane integrity, charge neutralization and stabilization of nucleic acids and retention of biological activity of nucleotides, and cofactor for numerous cytosolic and mitochondrial metabolic enzymes, however the molecular signals that rely on ₘMg²⁺ dynamics have not been fully elucidated. Remarkably, mammalian glycolytic end-product, L-lactate but not prokaryotic D-lactate acts as an activator that triggers a dynamic transfer of free Mg²⁺ ions between the ER and mitochondria to shape bioenergetics and cellular metabolism. The molecular switch that links ᵢMg²⁺ dynamics and cellular metabolism is unknown.

Unlike other organs, the liver plays a dominant role in glucose and lipid metabolism during increased energy intake and starvation. This cyclic process is distinctly controlled by hormones, ligand-gated GPCR signals, and ion channels. In hepatocytes, glucagon, vasopressin, and epinephrine are known to stimulate GPCR-linked ᵢCa²⁺ mobilization which is a prerequisite for glucose and fatty acid oxidation to generate heat and ATP production. Over decades, several mammalian Mg²⁺ transporters have been proposed to regulate cellular Mg²⁺ homeostasis, however its role in balancing carbohydrate and fat metabolism is ambiguous. Since the basal “free” Mg²⁺ dynamically controls ᵢ[Ca²⁺], any aberration to this signaling cascade may contribute to type II diabetes, obesity manifestation, hepatic NAFLD, progression to NASH, cirrhosis, and hepatocellular carcinoma. Nevertheless, how mammalian Mg²⁺ channels shape liver and adipose metabolism is unknown. The interrogation of ᵢMg²⁺ channels influence on long-term Western diet-induced hepatic metabolic signaling events leading to hepatic steatosis and obesity are extremely important.

There is a need for compositions and methods for modulation the ᵢMg²⁺ dynamics and whole-body metabolism.

### SUMMARY

The inventor has discovered that limiting mitochondrial Mg²⁺ uptake (ₘMg²⁺) prevents diet-induced obesity. The most abundant cellular divalent cations, Mg²⁺ (mM) and Ca²⁺ (nM-μM), antagonistically regulate divergent metabolic pathways with several orders of magnitude affinity preference. Although this phenomenon has been ascribed for several decades, the molecular queues remain unknown. Genetic ablation of mitochondrial Mg²⁺ channel Mrs2 enhanced fatty-acid transport into the mitochondria that evades adipose tissue expansion and fatty liver phenotype. Mrs2 deficiency restrains citrate efflux from the mitochondria that is a precursor of de novo lipogenesis. Elevated endogenous Mg²⁺ chelator citrate directly causes HIF-1α destabilization and subsequent biomass accumulation. Unbiased mRNA profiling identified that HIF-1α is linked to its capacity to enhance glycolysis and β-oxidation coupled thermogenesis exclusively in Mrs2 KO mice. Cooperatively, brown adipose tissue markers are markedly elevated in white adipose tissue (WAT) bed and confers weight-gain against long-term Western diet. Mrs2 channel blocker chloropentaamminecobalt (III) chloride (CPACC) has been identified as a treatment that lowers lipid droplet size in hepatocytes and prevents weight gain in obesity mouse model with normal liver function. The inventor contemplates that citrate is a negative regulator of HIF-1α-dependent signaling and is essential for cellular metabolism.

In certain aspects a modulator can be chloropentaammine cobalt(III) chloride (CPACC) or a derivative thereof.

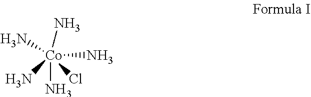

In other embodiments a CPACC derivative can have a chemical structure of Formula II.

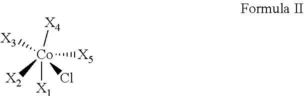

wherein X₁, X₂, X₃, X₄, and X₅ are independently selected from a halogen, amine, nitro, mercapto, hydroxyl, C1 to C4 alky, C1 to C4 heteroalkyl, alkoxy, alkylthio, or alkylamino. In certain aspects X₁, X₂, X₃, X₄, and X₅ are independently chlorine (Cl), bromine (Br), fluorine (F), or iodine (I). In certain aspects 1, 2, 3, 4, or all 5 of X₁, X₂, X₃, X₄, and X₅ are a halogen. In certain aspects 1, 2, 3, 4, or all 5 of X₁, X₂, X₃, X₄, and X₅ are chlorine.

Certain embodiments are directed to methods of ameliorating a metabolic syndrome comprising administering an effective amount of chloropentaammine cobalt (III) chloride (CPACC) or a variant thereof to a subject. The CPACC or a derivative thereof can be administered at a dose of between 10, 20, 30, 40, 50, 60, 70, 80, 90, 100, 150, 200, 250, 300, 350, 400, 450, to 500 μg or mg, including all values and ranges there between. The CPACC or a derivative thereof can be formulated as a tablet, a capsule, a concentrate, a powder, a drink, a baked good, chocolate, caramel, and/or snack. In certain aspects the CPACC or derivative thereof is formulated at a nutritional supplement. In certain aspects CPACC or a derivative thereof is administered orally. The CPACC or a derivative thereof can be administered once, twice, three times every day, week, month.

In certain embodiments the CPACC derivative has a chemical structure of Formula II.

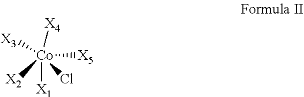

wherein X₁, X₂, X₃, X₄, and X₅ are independently selected from a halogen, amine, nitro, mercapto, hydroxyl, C1 to C4 alky, C1 to C4 heteroalkyl, alkoxy, alkylthio, or alkylamino. In certain aspects X₁, X₂, X₃, X₄, and X₅ are independently chlorine (Cl), bromine (Br), fluorine (F), or iodine (I). In a particular aspect 1, 2, 3, 4, or 5 of X₁, X₂, X₃, X₄, and X₅ are chlorine.

Certain embodiments are directed to methods of ameliorating diet-induced metabolic syndrome comprising administering chloropentaammine cobalt chloride (CPACC) or a variant thereof to a subject. The CPACC or a derivative thereof can be administered at a dose of between 10, 20, 30, 40, 50, 60, 70, 80, 90, 100, 150, 200, 250, 300, 350, 400, 450, to 500 μg or mg, including all values and ranges there between. The CPACC or a derivative thereof can be formulated as a tablet, a capsule, a concentrate, a powder, a drink, a baked good, chocolate, caramel, and/or snack. In certain aspects the CPACC or derivative thereof is formulated at a nutritional supplement. In certain aspects CPACC or a derivative thereof is administered orally. The CPACC or a derivative thereof can be administered once, twice, three times every day, week, month.

Certain embodiments are directed to methods for treating obesity comprising administering an effective amount of CPACC or a derivative thereof to an obese subject. The subject can have a body mass index (BMI) of 30 or greater. The subject can be diagnosed with pre-diabetes. The CPACC or a derivative thereof can be administered at a dose of between 10, 20, 30, 40, 50, 60, 70, 80, 90, 100, 150, 200, 250, 300, 350, 400, 450, to 500 μg or mg, including all values and ranges there between. The CPACC or a derivative thereof can be formulated as a tablet, a capsule, a concentrate, a powder, a drink, a baked good, chocolate, caramel, and/or snack. In certain aspects the CPACC or derivative thereof is formulated at a nutritional supplement. In certain aspects CPACC or a derivative thereof is administered orally. The CPACC or a derivative thereof can be administered once, twice, three times every day, week, month.

Certain embodiments are directed to methods for treating pre-diabetes comprising administering an effective amount of CPACC or a derivative thereof to a pre-diabetic subject. The CPACC or a derivative thereof can be administered at a dose of between 10, 20, 30, 40, 50, 60, 70, 80, 90, 100, 150, 200, 250, 300, 350, 400, 450, to 500 μg or mg, including all values and ranges there between. The CPACC or a derivative thereof can be formulated as a tablet, a capsule, a concentrate, a powder, a drink, a baked good, chocolate, caramel, and/or snack. In certain aspects the CPACC or derivative thereof is formulated at a nutritional supplement. In certain aspects CPACC or a derivative thereof is administered orally. The CPACC or a derivative thereof can be administered once, twice, three times every day, week, month.

Certain embodiments are directed to methods for treating diabetes comprising administering an effective amount of CPACC or a derivative thereof to a subject having diabetes. The CPACC or a derivative thereof can be administered at a dose of between 10, 20, 30, 40, 50, 60, 70, 80, 90, 100, 150, 200, 250, 300, 350, 400, 450, to 500 μg or mg, including all values and ranges there between. The CPACC or a derivative thereof can be formulated as a tablet, a capsule, a concentrate, a powder, a drink, a baked good, chocolate, caramel, and/or snack. In certain aspects the CPACC or derivative thereof is formulated at a nutritional supplement. In certain aspects CPACC or a derivative thereof is administered orally. The CPACC or a derivative thereof can be administered once, twice, three times every day, week, month.

Certain embodiments are directed to a composition comprising CPACC or a derivative thereof, the CPACC derivative having a chemical structure of Formula II

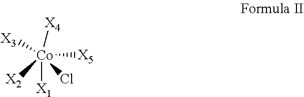

wherein X₁, X₂, X₃, X₄, and X₅ are independently selected from a halogen, amine, nitro, mercapto, hydroxyl, C1 to C4 alky, C1 to C4 heteroalkyl, alkoxy, alkylthio, or alkylamino.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1E. Metabolite Screen Identifies Lactate as activator/agonist for Mitochondrial Mg²⁺ Uptake. Dye loaded hepatocytes were stimulated with major cellular metabolites and mitochondrial oxidative phosphorylation (OXPHOS) inhibitors following baseline recording. (A) metabolites were tested for depletion of ER Mag-Green signal. (B) Depicts the reciprocal mobilization of free Mg²⁺ ion from ER to mitochondria was observed upon stimulation of Lactate (5 mM) in a temporal fashion. (C-E) Hepatocytes were simultaneously loaded with mitochondrial marker Mito-tracker Red and Mag-Green-AM to visualize the mitochondrial Mg²⁺ signal. (C) hepatocytes applied to low (5 mM) or high glucose (25 mM) concentrations. (D) Sodium lactate or lactic acid (5 mM). (E) Citrate (5 mM).

FIG. 2A-2F. Mitochondrial Mg²⁺ Transporter is Temperature Sensitive. Both ER-depletion and mitochondrial Mg²⁺ uptake was measured in hepatocytes at (A, B) 37° C.; (C, D) 37° C. and 25° C.; and (E, F) 25° C. to 40° C.

FIG. 3A-3B. ER-Dependent Efflux of Free Mg²⁺ Promotes Mitochondrial Mg²⁺ Uptake. Primary hepatocytes were treated with MCT1 or MCT1/2 inhibitors for 60 minutes. (A) ER depletion and (B) mitochondria uptake.

FIG. 4A-4M Lowering [Mg²⁺]ₘ prevents diet induced obesity. (A) Body weight change over a 30-week diet period. Mean+/−SEM. Data fitted with a Gompertz curve. Mean+/−SEM. (n=4-16 mice). (B) Weight gained over the dietary course. Mean+/−SEM. One-way ANOVA with Tukey's multiple comparison. ****=P<0.0001, **=P<0.01, n.s.=not significant. (C) Liver, heart, and kidney weights after 1 year diet period. One-way ANOVA with Tukey's multiple comparison. ‡=Not significant, multiple comparisons are done the same as the liver. n=3-4 mice (organs) per group. ****=P<0.0001, n.s.=not significant. Percent lean mass (D) and fat mass (E) of indicated groups. One-way ANOVA with Tukey's multiple comparison. n=3-4 mice (organs) per group. ****=P<0.0001, ***=P<0.001, **=P<0.01, *=P<0.05, n.s.=not significant. (F) Measurement of food intake of 1 year CD or WD fed mice during metabolic screening with Promethion system. Mean+/−SEM. Water consumption (G) and locomotion (H) of mice during metabolic screening with Promethion system. Mean+/−SEM. (I) Plasma levels of cholesterol after mice subjected to 16 hrs of fast followed by 4 hrs of fed state. One-way ANOVA with Tukey's multiple comparison. n=4-5 mice per group. **=P<0.01, n.s.=not significant. (J) Energy expenditure of WT or Mrs2^{-/-} mice fed CD and WD mice for 1 year. Data fit with a fixed-frequency sinusoidal equation y=B+A sin(0.25x+j). (WT; CD, n=5 and WD, n=4), (Mrs2^{-/-}; CD, n=5, WD, n=4) mice per group. (K) The bar graph depicts normalized energy expenditure data derived from four different groups. Energy expenditure of these mice were individually measured. ****=P<0.0001, n.s.=not significant. (L) Respiratory exchange ratio (RER) of WT or Mrs2^{-/-} mice fed CD and WD mice for 1 year. Data fit with a fixed-frequency sinusoidal equation y=B+A sin(0.25x+j). (WT; CD, n=5 and WD, n=4), (Mrs2^{-/-}; CD, n=5, WD, n=4) mice per group. (M) The bar graph depicts normalized RER amplitude data derived from four different groups. RER of these mice were individually measured. ****=P<0.0001, n.s.=not significant.

FIG. 5A-5F. Mrs2 deletion prevents diet-induced liver damage, vascular density loss, and hepatocellular carcinoma burden. (A) ALT (alanine transaminase) levels in the plasma after 1 year diet period. One-way ANOVA with Tukey's multiple comparison. n=7-8 mice. (B) HCC incidence in representative liver tissue sections stained for Hematoxylin & eosin (H&E, top) and Masson's trichrome (bottom) from WT and Mrs2^{-/-} Western Diet (WD) fed after 1 year diet period. n=3 mice per group. (C) Immunohistochemical analysis of sinusoidal vascular density in liver samples obtained from WD fed WT (left) and Mrs2^{-/-} mice after 1 year diet period. n=3 mice per group. (D) Quantification of representative kidney sections stained for Masson's Trichrome from WT and Mrs2^{-/-} chow diet or Western diet-fed after 1 year diet period. n=3 mice per group. Dot plot indicates the quantification of collagen staining. Randomly five slides were used for quantification. Mean±SEM. **=P<0.01 ****=P<0.0001, n.s.=not significant. (E and F) Blood glucose and plasma insulin levels in fasting or fed states of CD or WD mice from two genetic backgrounds. ****=P<0.0001, **=P<0.01, n.s.=not significant.

FIG. 6A-6C. Blunting ₘMg²⁺ dynamics reprograms whole animal RNA transcripts that enhances adipocyte browning and hyper oxidative phosphorylation against Western diet. (A) Variability between biological replicates (within groups) for Western diet fed mice. % variability indicates the proportion of protein-coding genes that are uniquely expressed in a single replicate within the indicated group. Mean±SEM. (B) Venn diagram depicts the differentially expressed protein-coding genes between WT and Mrs2^{-/-} mice in Western diet or chow diet conditions. Liver tissue (top) and iWAT (bottom). H) Heatmap depicting relative mRNA abundance of Ucp1 and Lep in iWAT from WT and Mrs2^{-/-} mice fed either chow diet or Western diet. n=3 mice per group. (C) Mitochondria per field.

FIG. 7A-7I. Enhancement of mitochondrial fatty acid flux and bioenergetics was observed in Western diet fed mice lacking Mrs2 channel. (A-B) Quantification of hepatocyte lipid droplet size and ratio of lipid droplet to mitochondria area in hepatocytes isolated from 1 year diet period of WT and Mrs2^{-/-} mice. One-way ANOVA with Tukey's multiple comparison. n=3-4 mice. Minimum of 5 images per mouse were used for quantification. ****=P<0.0001, **=P<0.01, n.s.=not significant. (C) Spatial overlap and intensity profiles of mitochondrial co-localization of BODIPY and TMRE signals. n=3 isolations per group. Pearson's coefficient was analyzed to determine the colocalization. (D) Western analysis of mitochondrial carnitine palmitoyl transferase 1A and MCU complex, MCU and MICU1 protein abundance in liver tissues harvested from the 1-year diet period of WT and Mrs2^{-/-} mice. n=4 mice per group. ****=P<0.0001, **=P<0.01, *=P<0.05, n.s.=not significant. Assessment of MCU-mediated ₘCa²⁺ uptake (E), retention capacity (F), DΨₘ maintenance by hepatocytes isolated from the 1-year diet period of WT and Mrs2^{-/-} mice. n=4 mice per group (G). ***=P<0.001, **=P<0.01, n.s.=not significant. (H) Mitochondrial oxygen consumption rate in hepatocytes isolated from the 1-year diet period of WT and Mrs2^{-/-} mice using Seahorse platform and normalized to total protein content. n=3 mice per group. (I) Bar charts depict basal and maximal respiration and proton leak from the above traces. Each data point represents individual wells from three different hepatocyte isolations of four groups. ****=P<0.0001, n.s.=not significant.

FIG. 8A-8J. Elevation of plasma citrate and higher rate of mitochondrial efflux was blunted in Western diet Mrs2 KO mice. (A) Plasma citrate levels were measured in WT and Mrs2 KO mice fed with chow or Western diet. Mean±SEM. n=4-7 mice per group. **=P<0.01, n.s.=not significant. (B) Representative traces depict mitochondrial citrate efflux in WT or Mrs2 KO hepatocytes. The change was measured as a function of Mag-green fluorescence quenching. Permeabilized hepatocytes were suspended with Mag-green and pulsed with a bolus of 1 mM MgCl₂. After steady state, oxaloacetate was added as a substrate for mitochondrial citrate production and efflux was monitored as a Mag-Green fluorescence reduction. (C) Quantification mito-Citron1 fluorescence dynamics after glucose stimulation. (D) Measurement of mito-Citron1 dynamics in WT or Mrs2 KO hepatocytes after stimulation with 20 mM glucose. Mean±SEM. n=3. ****=P<0.0001. (E) Measurement of cytosolic-Citron1 dynamics in WT or Mrs2 KO hepatocytes after stimulation with 20 mM glucose. Mean±SEM. n=3. ****=P<0.0001. (F) Glucose-induced cytosolic citrate changes in WT hepatocytes and hepatocytes expressing Mrs2 channel dead mutant adenovirus5. Bar graph depicts the quantification of cyto-Citron1 fluorescence change after 20 mM glucose stimulation. Mean±SEM. n=3. ****=P<0.0001. (G) Normalized Slc25al gene expression in isolated hepatocytes from CD or WD-fed WT and Mrs2^{-/-} mice. Data represented as % of WT CD, mean±SEM. n=3 per group. ****=P<0.0001, n.s.=not significant. Measurement (left) and quantification (right) of mito-Citron1 (H) or cyto-Citron1 (I) dynamics in HepG2 cell line infected with either Ad-RFP or Ad-mutant-Mrs2-RFP. 20 mM glucose stimulation. Mean±SEM. n=3. ****=P<0.0001. (J) Mitochondrial Mg²⁺ uptake assay coupled with citrate efflux assay in WT or Mrs2^{-/-} hepatocytes. The change was measured as a function of Mag-green fluorescence quenching. Permeabilized hepatocytes were suspended with Mag-green and pulsed with a bolus of 1 mM MgCl₂. After steady state, oxaloacetate was added as a substrate for mitochondrial citrate production and citrate efflux was monitored as Mag-Green fluorescence reduction.

FIG. 9A-9R. Pleiotropic factor citrate destabilizes HIF-1a further its role as endogenous chelator of Mg²⁺ and de novo lipogenesis precursor. (A) HIF-1a stabilization in hepatocytes derived from WT or Mrs2 KO mice. Hepatocytes were treated with LPS or CoCl₂ for ₆ hours. Total lysates were subjected to Western blot analysis to determine HIF-1a protein abundance. n=3 independent hepatocyte isolations. (B and C) Image J analysis of HIF-1a protein abundance. ****=P<0.0001, n.s.=not significant. (D) Targeted metabolite screen identified citrate as an endogenous HIF-1a destabilizer. WT hepatocytes were treated with PHD inhibitor FG-4592 (100 mM) for 6 hours with or with metabolites. Total cell lysates were probed for HIF-1a protein abundance. Bottom panel depicts Image J analysis of protein abundance. (n=2). (E) Dose curve for citrate-induced HIF-1a destabilization. Right panel shows the normalized protein abundance. (n=2). (F) Effect of citrate on constitutively stable HIF-1a mutant protein. Top panel shows citrated induced HIF-1a degradation in the presence of various doses of citrate. (n=2). Bottom panel depicts the effect of citrate on oxygen insensitive HIF-1a. (n=2). (G) Effect of citric acid and its derivatives on HIF-1a stabilization. (n=2). Bottom panel shows the effect of citrate on HIF-1a stabilization in hepatocytes derived from WT or Mrs2 KO mice (n=2). (H) Effect of citrate on FG-4952, DMOG, or CoCl₂-dependent HIF-1a stabilization in WT hepatocytes (n=2). (I) Effect of mitochondrial pyruvate transporter blocker UK5099 or ᵢMg²⁺ chelator EDTA on HIF-1a stabilization. (n=2). (J) Effect of MgCl₂ supplementation on HIf-1a stabilization under normoxia. (n=2). (K) Effect of TCA cycle citrate precursor OAA or a-KG on HIF-1 destabilization. (n=2). (L) Intracellular accumulation of citrate elicits HIF-1a destabilization. Right panel depicts reciprocal action of citrate on FG-4592-dependent HIF-1a stabilization. (n=3). (N-P) FG-4592, CoCl₂ or DMOG induced HIF-1a stabilization in WT hepatocytes were treated for 6 hours and total lysates were subjected to Western blot analysis to determine HIF-1a protein abundance. n=2-3 independent hepatocyte isolations. (Q) FG-4592 mediated HIF1a stabilization is destabilized by citrate in a time-dependent fashion. As indicated, citrate (10 mM) was added to FG-4592 (100 mM) hepatocytes. n=2 independent hepatocyte isolations. (R) FG-4592 treated hepatocytes were permeabilized and challenged with citrate at various concentration in vitro. Quantification of HIF1a protein abundance from the Western blot. n=2 independent experiments.

FIG. 10A-10O. Pharmacologic blockade of Mrs2-mediated Mg²⁺ uptake results in hepatocyte lipid droplet size reduction and improves bioenergetics. (A) Chemical structures of Chloropentaamminecobalt (III) chloride [Co(NH₃)₅Cl]Cl₂(CPACC) and Hexaamminecobalt (III) chloride [Co(NH₃)₆]Cl₃. (B) Permeabilized hepatocytes were pulsed with 1 mM MgCl₂ or in combination with various concentrations of CPACC. Representative traces show bath [Mg²⁺] (f.a.u). Mean±SEM. n=3. (C, E, G) Permeabilized hepatocytes were pulsed with 1 mM MgCl₂ or in combination with various concentrations of chloropentaammine Co(III) chloride, Ru(III) hexaammine chloride, chloropentaammine Ru(III) chloride, or cobalt(III) hexaammine chloride. Representative traces show bath [Mg²⁺] (f.a.u). Mean±SEM. n=3-4. *=P<0.05, **=P<0.01, ***=P<0.001, *** =P<0.0001, n.s.=not significant. (D) Calculation of dose-dependent inhibition of Mrs2-mediated ₘMg²⁺ uptake by CPACC. n=3. (H) Ca²⁺ dynamics in control (n=1) or 10 μM CPACC (n=3) treated HeLa cells measured using spectrofluorometry. Quantification of the rate of ₘCa²⁺ uptake from linear regression analysis (bottom). (I) Cellular uptake of CPACC in WT hepatocytes. Intracellular Co(III) was measured by atomic absorption spectroscopy. Mean±SEM. n=3. **=P<0.01. (J) Cytotoxicity Curve. (K) Distribution of lipid droplets in hepatocytes following CPACC treatment. Data are the number of mean Mean±SEM. n=3. (L) Quantification of lipid droplet size hepatocytes following CPACC treatment. n=3-4. ****=P<0.0001.

FIG. 11A-11J. Limiting Mrs2-mediated Mg²⁺ uptake by CPACC promotes adipocyte browning in vivo. (A) Quantification of lipid droplet sizes in hepatocytes following CPACC treatment (50 mM, 48 hours). n=3-4 independent experiments. ****=P<0.0001. (B) Measurement of DYₘ following CPACC treatment (50 mM, 48 hours). n=3-4 independent experiments. Over 30 hepatocytes were used for the TMRE intensity analysis. n.s.=not significant. Size distribution of lipid droplets in hepatocytes untreated (C) following CPACC treatment (D). The area of droplet size was quantified from multiple cells. n=3. (E) Mitochondrial oxygen consumption rate in WT hepatocytes with or without CPACC treatment. n=3 independent experiments. 8 wells per trial. Mean±SEM. (F) Bar charts depict basal respiration, maximal respiration, and proton leak from the FIG. 8F traces. Mean±SEM. n=3-6. *=P<0.05, n.s.=not significant. (G) Traces represent extracellular acidification rate in control or CPACC treated WT hepatocytes. Bar charts show basal and glycolytic reserve upon challenge with mitochondrial inhibitors. Mean±SEM. n=3-6. ***=P<0.001, **=P<0.01, *=P<0.05, n.s.=not significant. (H) WT mice were subjected to HFD for 14 weeks followed by CPACC (20 mg/kg) or vehicle for additional six weeks via i.p. every three days. The body weight was measured weekly. Mean±SEM. n=3 mice per group. (I) Body weight change during the 6-week period of CPACC treatment. Mean±SEM. n=3 mice per group. *=P<0.05. (J) Bar graph depicts plasma ALT levels of WT HFD with or without CPACC treatment. n=5 mice/group. *=P<0.05.

FIG. 12. Mice fed a Western diet were treated with vehicle or CPACC after 20 weeks diet and monitored for an additional 10 weeks. Dotted line represented the expected (mathematical modeling) body weight and the solid line represents the linear model fit of the observed body weight. Data represented as mean+/− SEM n=4 per group. Right: Change in body weight during treatment period (week 30 weight minus week 20 weight).

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be examples of that embodiment, and not intended to intimate that the scope of the disclosure, including the claims, is limited to that embodiment.

It has been established that the four major bulky cations Na⁺, K⁺, Mg²⁺, and Ca²⁺ control numerous cellular functions including membrane polarization, bioenergetics, metabolism, transcription, and proliferation. During cellular activation, the highly compartmentalized Ca²⁺ ions rapidly raise cytosolic concentration to initiate ‘on’ reactions. Consequently, the antagonistic cellular Mg²⁺ dynamics carry out the offset reactions that are in the in the form of bound or free ionic states. Although this concept was proposed for several decades, ᵢMg²⁺ dynamics and its intrinsic role remains unknown. Cellular Mg²⁺ is the intrinsic component of numerous metabolic enzymes, phosphometabolites, and metabolic precursors such as citrate. For instance, GPCR, Nicotinic, and NMDA receptors, L-type Ca²⁺ channels, and ion pumps activity were modulated by Mg²⁺ and aberrations led to several prevalent diseases such as hypertension, heart disease, diabetes, ischemia/reperfusion, preeclampsia, cancer, COPD, asthma, migraine, X-linked T-cell immunodeficiency, neurological diseases, Parkinson's disease, and Alzheimer's disease. The precise mechanism of action remains a mystery and is often surrounded by a matter of controversy.

Glycolytic end-product L-lactate acts as an activator of ER Mg²⁺ release, and the elevated free ᵢMg²⁺ was subsequently taken up by mitochondria through a highly conserved Mg²⁺ selective Mrs2 transport machinery. The physiological function of Mrs2-mediated Mg²⁺ uptake and its effect on bioenergetics and whole-body metabolic consequences thereof are unknown. To recognize the contribution of ₘMg²⁺ in physiology and disease context, a mouse model of long-term Western diet induced metabolic disease progression was used, providing various salient findings that were undiscovered. To identify the pathways that facilitate diet-induced metabolic disorders, control and global Mrs2 null mice were fed for over 52 weeks with Western diet. The findings reveal both qualitatively and quantitatively different mechanisms in diet regulated metabolic syndrome that is Mg²⁺ dependent. The gross anatomical phenotype of Mrs2 KO mice exhibited no body weight gain, near complete ablation of energy storage expansion and prevention of spontaneous hepatocellular carcinoma incidence, retention of sinusoidal microvascular density with normal liver function during the course of Western diet regime. Remarkably, Mrs2 KO mice had consumed more food with the retention of normal circadian rhythm in the form of energy expenditure otherwise this phenomenon is disrupted in the WT WD mice. Having observed such a significant physiological outcome, it is contemplated that the common feature underlying these diverse regulatory mechanisms including thermogenic activation, hyper glucose and fatty oxidation, and oxidative phosphorylation events were under low [Mg²⁺]ₘ scenario. Histological, biochemical, and global transcriptomic regulation analyses fully support the absence of metabolic syndrome phenotype in Mrs2 KO mice.

It has also been found that a notable reprograming of energy storage organ transcript profile favors thermogenesis, glycolysis, fatty acid oxidation, and oxidative phosphorylation in Mrs2 KO that memory was enhanced upon Western diet supplementation. The data indicate that the retrograde signal that emanates from mitochondria controls transcriptional profiles through Mg²⁺ dependent manner. A marked suppression of mitochondrial citrate efflux and subsequent stabilization of HIF-1α was identified in Mrs2 KO mice indicating that enhanced HIF-1α-dependent glucose uptake, glycolysis, and vascularization (oxygenation) are essential for oxidative phosphorylation. This new finding sheds light on how intracellular divalent cation dynamics control diet-induced metabolic dysregulation and disease progression. For instance, glucose clearance and oxidation are suppressed in diabetes and other metabolic disease conditions. It has been proven that aerobic or ischemic-mediated HIF-1α stabilization exerts protection against coronary artery disease which is consistent with the finding that lowering ₘMg²⁺ prevents Western diet induced obesity in rodents. Aerobic stabilization of HIF-1α transcriptionally controls several monovalent, divalent cation channels, and mitochondria localized SOD2 that prevents oxidative stress and hypertension implying the influence of ₘMg²⁺ and HIF-1α-dependent signaling in cellular metabolism.

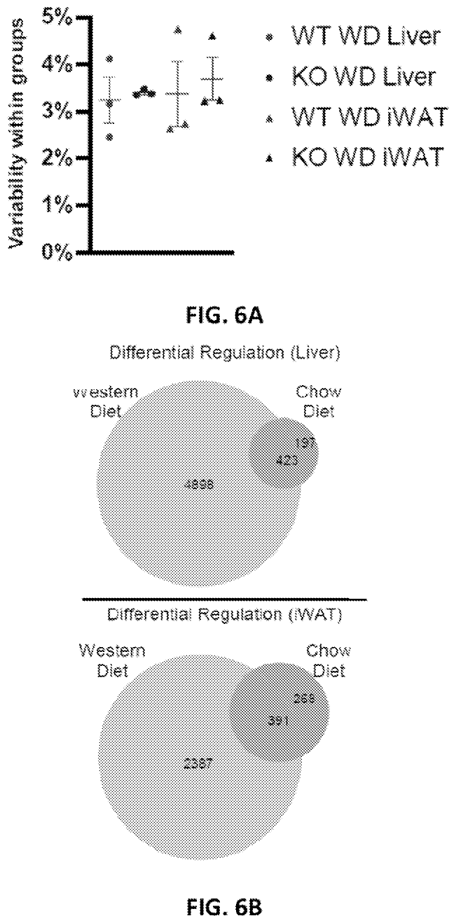

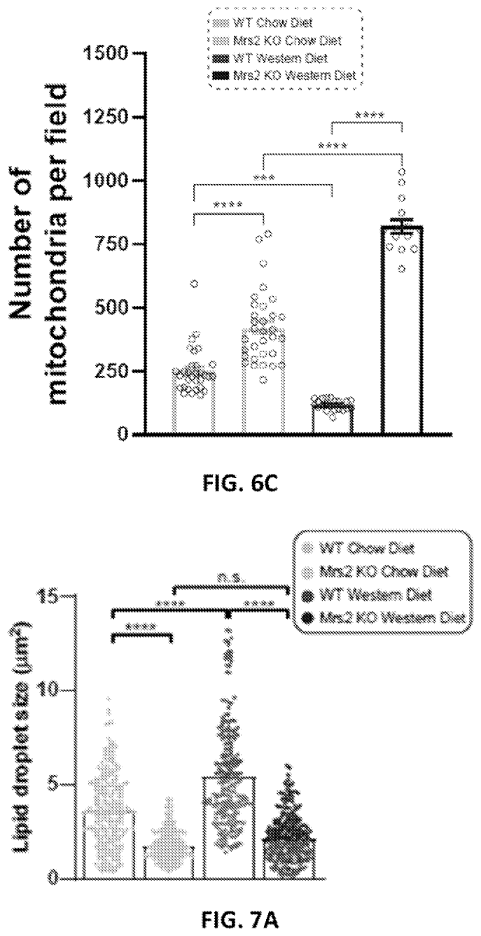

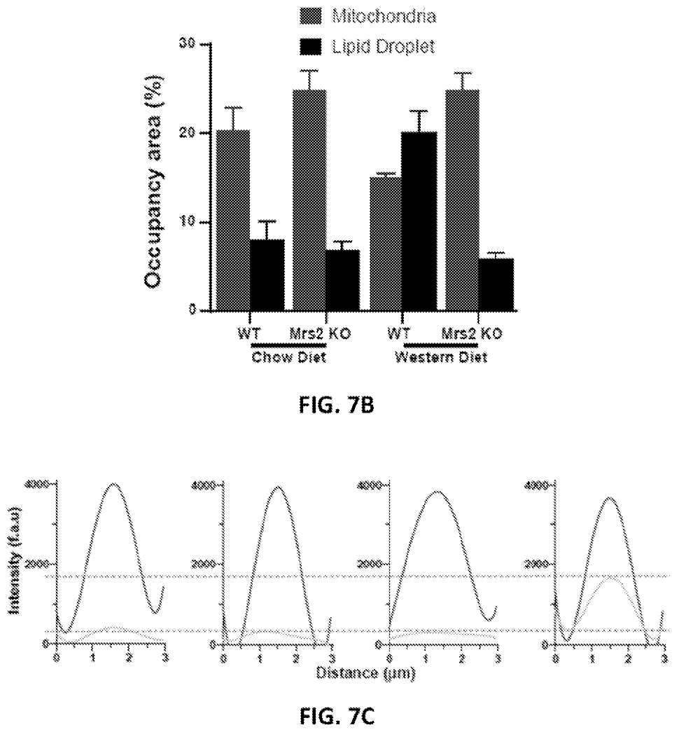

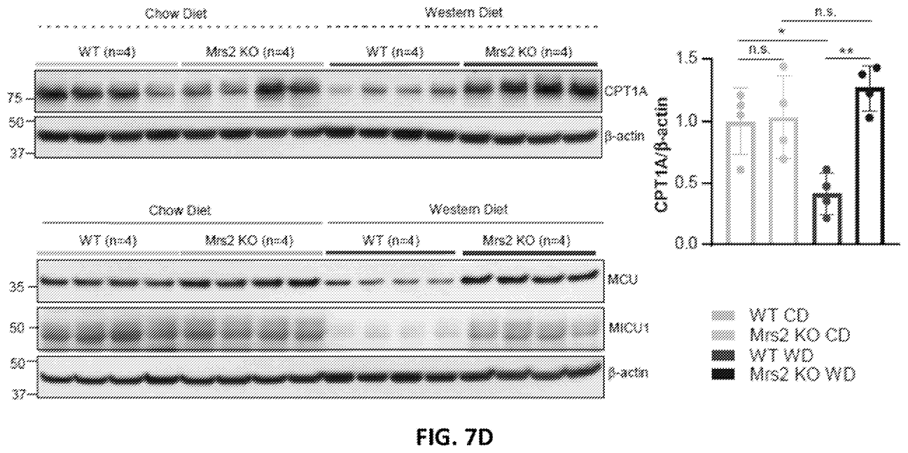

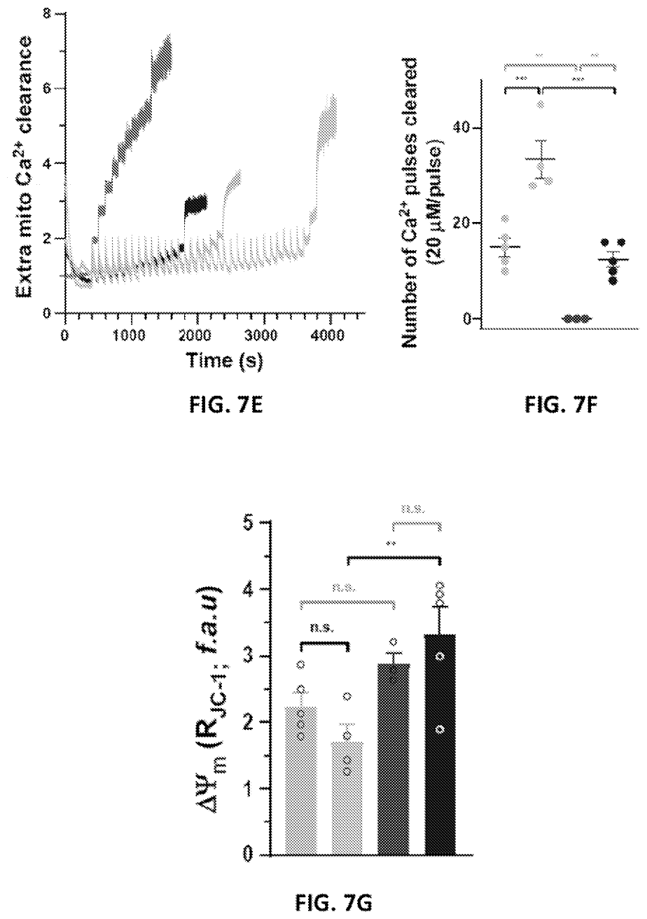

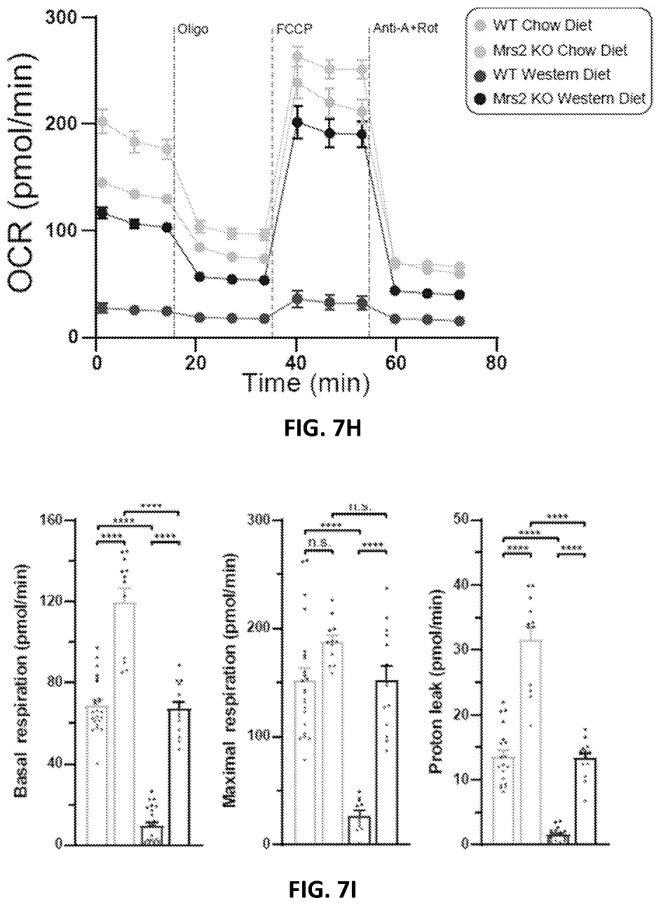

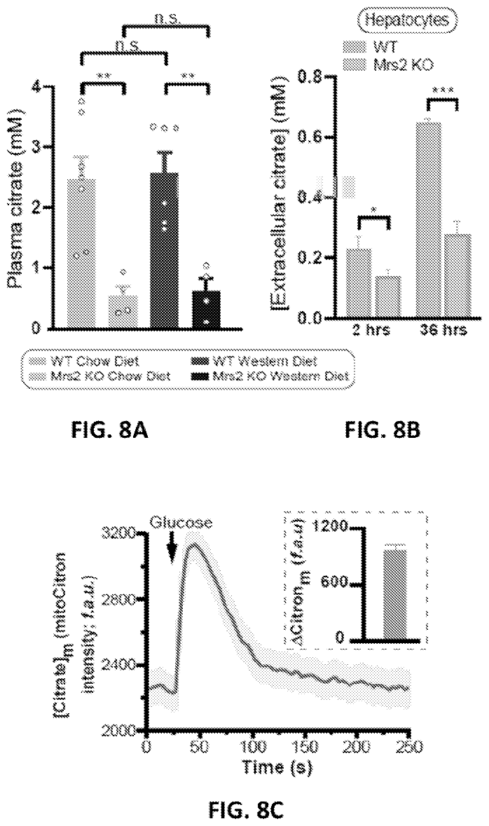

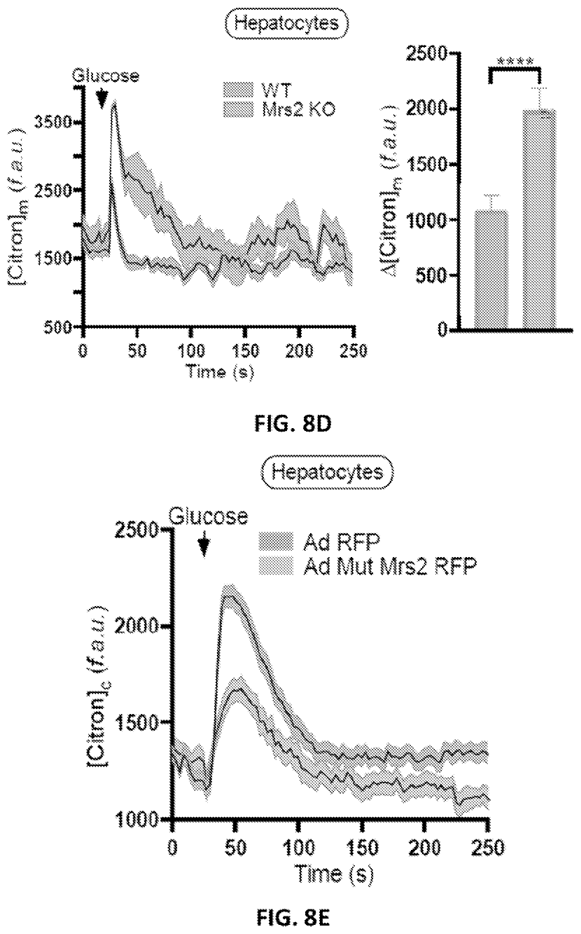

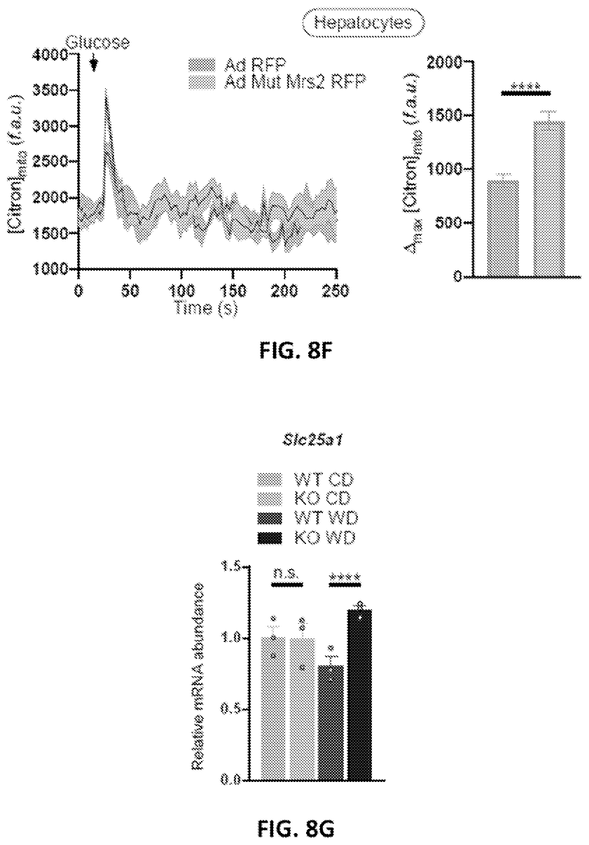

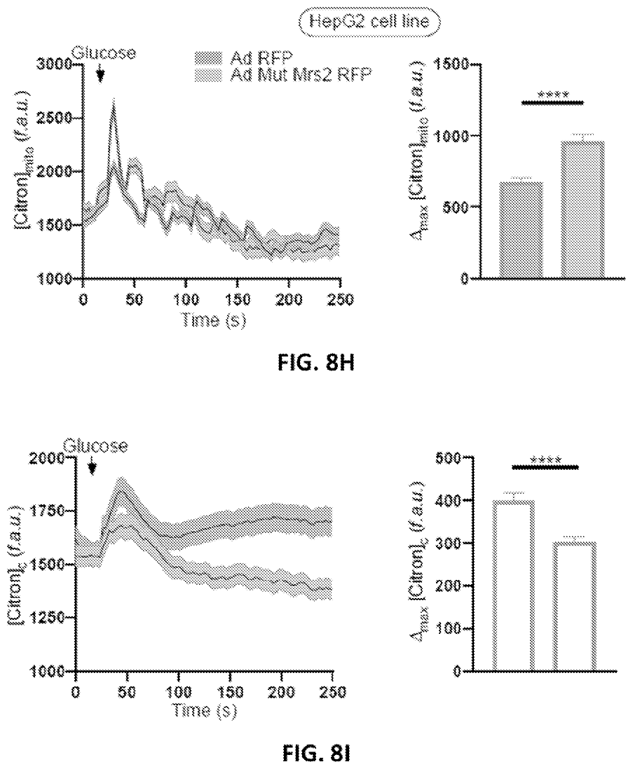

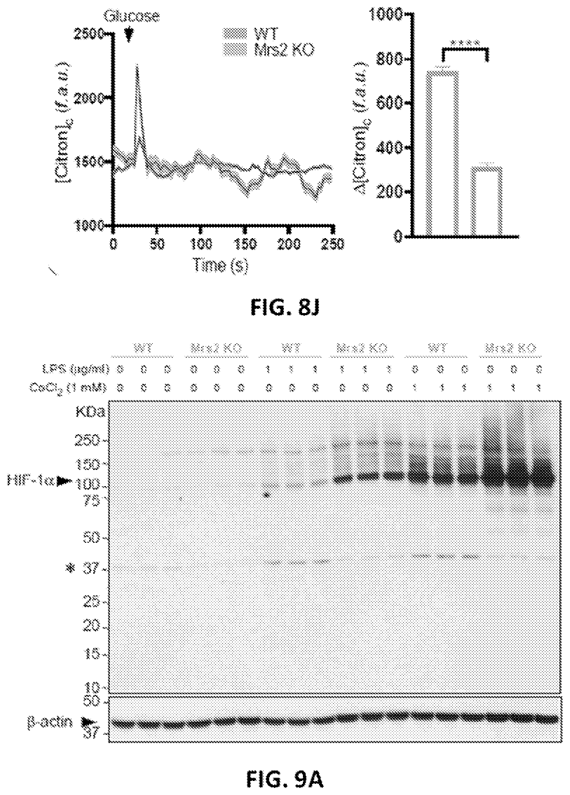

The findings reveal that blockade of mitochondrial Mg²⁺ flux by both genetic and pharmacologic interventions diminished hepatic lipid droplet occupancy (FIG. 7 and FIG. 8) and enhances thermogenic adipocytes population (FIG. 6). It is noteworthy that Cobalt derivative (CPACC) inhibits ₘMg⁺ uptake at lower micromolar range in hepatocytes that promotes hepatic lipid droplet size reduction and parallelly facilitates partial stabilization of HIF-1α as well in cell culture suggesting a dual target prevents diet induced adipocyte hypertrophy and obesity (FIG. 8). Since CorA channel interacts with the fully hydrated Mg²⁺ ion, it was also hypothesized that ammines group of the divalent and trivalent cations mimic the size and shape of a hydrated Mg²⁺ cation that exerts the inhibition of Mg²⁺ ion permeation. Nevertheless, this proof-of-concept approach will aid to design Mg²⁺ channel modulators that buffer Western diet induced adipocyte expansion. Taken together, the findings reveal a mechanistic link between ₘMg²⁺ dynamics and cellular energy metabolism in mammals. The experimental findings identified that Mrs2 blocker CPACC lowers lipid accumulation in the liver and prevents weight gain against Western diet/high fat diet.

In certain aspects the Magnesium transporter MRS2 homolog has the amino acid sequence

(SEQ ID NO: 1)
MECLRSLPCLLPRAMRLPRRTLCALALDVTSVGPPVAACGRRANLIGRS
RAAQLCGPDRLRVAGEVHRFRTSDVSQATLASVAPVFTVTKFDKQGNVT
SFERKKTELYQELGLQARDLRFQHVMSITVRNNRIIMRMEYLKAVITPE
CLLILDYRNLNLEQWLFRELPSQLSGEGQLVTYPLPFEFRAIEALLQYW
INTLQGKLSILQPLILETLDALVDPKHSSVDRSKLHILLQNGKSLSELE
TDIKIFKESILEILDEEELLEELCVSKWSDPQVFEKSSAGIDHAEEMEL
LLENYYRLADDLSNAARELRVLIDDSQSIIFINLDSHRNVMMRLNLQLT
MGTFSLSLFGLMGVAFGMNLESSLEEDHRIFWLITGIMFMGSGLIWRRL
LSFLGRQLEAPLPPMMASLPKKTLLADRSMELKNSLRLDGLGSGRSILT
NR

In certain aspects a metabolic modulator can be chloropentaammine cobalt (III) chloride (CPACC) (Formula I) or a derivative thereof.

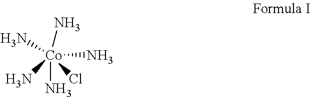

In other embodiments a CPACC derivative can a chemical structure of Formula II.

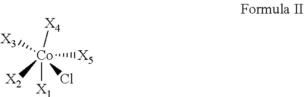

wherein X₁, X₂, X₃, X₄, and X₅ are independently selected from a halogen, amine, nitro, mercapto, hydroxyl, C1 to C4 alky, C1 to C4 heteroalkyl, alkoxy, alkylthio, or alkylamino. In certain aspects X₁, X₂, X₃, X₄, and X₅ are independently chlorine (Cl), bromine (Br), fluorine (F), or iodine (I). In certain aspects 1, 2, 3, 4, or all 5 of X₁, X₂, X₃, X₄, and X₅ are a halogen. In certain aspects 1, 2, 3, 4, or all 5 of X₁, X₂, X₃, X₄, and X₅are chlorine.

As used herein, the term “nitro” means-NO₂; the term “halo” or “halogen” designates —F, —Cl, —Br or —I; the term “mercapto” means —SH, and the term “hydroxy” means —OH.

The term “alkyl,” by itself or as part of another substituent, means, unless otherwise stated, a linear (i.e. unbranched) or branched carbon chain, which may be fully saturated, mono-or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH. (Me), —CH₂CH₃(Et), —CH₂CH₂CH₃(n-Pr), —CH(CH₃)₂ (iso-Pr), —CH₂CH₂CH₂CH₃ (n-Bu), —CH(CH₃)CH₂CH₃(sec-butyl), —CH₂CH(CH₃)₂(iso-butyl), —C(CH₃)₃(tert-butyl), —CH₂C(CH₃)₃(neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, trichloromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, and —CH₂CH₂OC(O)CH₃.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, or alkylamino.

The term “alkoxy” means a group having the structure —OR′, where R′is an optionally substituted alkyl or cycloalkyl group. The term “heteroalkoxy” similarly means a group having the structure —OR, where R is a heteroalkyl or heterocyclyl.

The term “amino” means a group having the structure —NR′R″, where R′ and R″ are independently hydrogen or an optionally substituted alkyl, or heteroalkyl. The term “amino” includes primary, secondary, and tertiary amines.

The term “oxo” as used herein means an oxygen that is double bonded to a carbon atom.

### Pre-Diabetes Syndrome

Pre-diabetes is the state in which some but not all of the diagnostic criteria for diabetes are met, including impaired fasting glycemia or impaired fasting glucose (IFG) and impaired glucose tolerance (IGT). IFG refers to a condition in which the fasting blood glucose is elevated above what is considered normal levels but is not high enough to be classified as diabetes mellitus. Fasting blood glucose levels are in a continuum within a given population, with higher fasting glucose levels corresponding to a higher risk for complications caused by the high glucose levels. IFG is defined as a fasting glucose that is higher than the upper limit of normal, but not high enough to be classified as diabetes mellitus. Some patients with impaired fasting glucose can also be diagnosed with IGT as described below, but many have normal responses to a glucose tolerance test.

IFG is considered a pre-diabetic state, associated with insulin resistance, increased mortality, and increased risk of cardiovascular pathology, although of lesser risk than IGT (Barr et al. Circulation. 2007, 116(2):151-157). There is a 50% risk over 10 years of progressing to overt diabetes, but many newly identified IFG patients progress to diabetes in less than three years (Nichols et al. Diabetes Care. 2007, 30(2):228-233).

IGT is a pre-diabetic state of dysglycemia, that is associated with insulin resistance and increased risk of cardiovascular pathology. IGT may precede type 2 diabetes mellitus by many years. IGT is also a risk factor for mortality (Nichols et al. Diabetes Care. 2007, 30(2):228-233).

Following the ADA criteria, pre-diabetes can be diagnosed with a blood test with any of the following results: (1) Fasting blood sugar (glucose) level from 100-125 mg/dl (5.6-6.9 mM); (2) A blood sugar level of 140 to 199 mg/dL (7.8 to 11.0 mM) two hours after ingesting the standardized 75 gram glucose solution in the glucose tolerance test; (3) Glycated hemoglobin between 5.7 and 6.4%.

Diabetes can be diagnosed with a blood test with any of the following results: (1) Fasting blood sugar (glucose) level≥126 mg/dl (7.0 mM); (2) A blood sugar level≥200 mg/dL (11.1 mM) two hours after ingesting the standardized 75 gram glucose solution in the glucose tolerance test; (3) Glycated hemoglobin≥6.5%; (4) Symptoms of hyperglycemia and casual plasma glucose≥200 mg/dl (11.1 mM).

### Obesity

Obesity refers to a condition where excessive fat accumulates within the body. In general, when a person's body mass index (BMI) is greater than 30, they are diagnosed as obese. Body mass index (BMI) is a widely used method for estimating body fat mass and is an accurate reflection of body fat percentage in the majority of the adult population. BMI is calculated by dividing the subject's mass by the square of his or her height, typically expressed either in metric or US “Customary” units as kg/m² or pounds×703/inches². A person with a BMI of 30.0 or greater is defined as obese, with higher BMI values being further classified as severe obesity (35.0 to 40), morbid obesity (40.0 to 45), and super obese (BMI≥45).

Obesity is caused by an energy imbalance over a long period when an excessive amount of calories are ingested with respect to the amount of energy being expended. Treatment of obesity normally requires behavior therapy as well as a reduction of calories ingested and/or an increase in the amount of calories expended.

Low levels of the protein adiponectin have been associated with a higher risk of developing metabolic syndromes such as obesity. In certain aspects the methods described herein result in the upregulation of adiponectin. Adiponectin (also referred to as GBP-28, apM1, AdipoQ and Acrp30) is a 244 amino acid protein that in humans is encoded by the ADIPOQ gene. It is a protein hormone that modulates a number of metabolic processes, including glucose regulation and fatty acid oxidation. Adiponectin is secreted into the bloodstream from adipose tissue and also from the placenta in pregnancy (Chen et al. Diabetalogica. 2006, 49(6):1292-1302). In the bloodstream, adiponectin accounts for approximately 0.01% of all plasma protein at around 5-10 μg/mL and is very abundant in plasma relative to many hormones. Levels of the hormone are inversely correlated with body fat percentage in adults, while the association in infants and young children is less clear (Ukkola and Santaniemi. J Mol Med. 2002, 80(11):696-702).

Transgenic mice with increased adiponectin show impaired adipocyte differentiation and increased energy expenditure associated with protein uncoupling (Bauche et al. Endocrinology. 148(4):1539-1549). The hormone plays a role in the suppression of the metabolic derangements that may result in type 2 diabetes, obesity, atherosclerosis, non-alcoholic fatty liver disease (NAFLD) and an independent risk factor for metabolic syndrome (Diez and Iglesias. Eur J Endocrinol. 2003, 148(3):293-300; Ukkola and Santaniemi. J Mol Med. 2002, 80(11):696-702; Renaldi et al. Acta Med Indones. 2009, 41(1):20-24). Adiponectin in combination with leptin has been shown to completely reverse insulin resistance in mice (Yamauchi et al. Nat Med. 2001, 7(8):941-946). Levels of adiponectin are reduced in diabetics compared to non-diabetics. Weight reduction significantly increases circulating levels (Coppola et al, Int J Cardiol. 2008 134(3):414-416).

### Formulation and Administration

CPACC and derivatives thereof can be administered to a subject either orally, parenterally (e.g., intravenously, intramuscularly, or subcutaneously), intraperitoneally, or locally (for example, powders, ointments or drops). In certain aspects the compounds are provided in a nutritional supplement formulation. A nutritional supplement formulation can be in any form, e.g., liquid, solid, gel, emulsion, powder, tablet, capsule, or gel cap (e.g., soft or hard gel cap). A nutritional supplement formulation typically will include one or more compositions that have been purified, isolated, or extracted (e.g., from plants) or synthesized, which are combined to provide a benefit (e.g., a health benefit in addition to a nutritional benefit) when used to supplement food in a diet.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Suspensions, in addition to the active compound(s), may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Dosage forms for topical administration of ursolic acid and resveratrol include ointments, powders, sprays and inhalants. The compound(s) are admixed with a physiologically acceptable carrier, and any preservatives, buffers, and/or propellants that may be required.

For the compounds of the present invention, alone or as part of a therapeutic or supplement composition, the doses are between about 1, 100, 200, 300, 400, 500, 600 to 500, 600, 700, 800, 900, 1000 μg or mg, preferably between 10 and 500 μg or mg. In certain aspects, a composition is administered 1, 2, 3, 4, 5, 6, 7, 8, 9, or 10 times per day, week or month. In certain aspects, the compounds are administered once every 1, 2, 3, 4, 5, 6, or 7 days.

The term “effective amount” means an amount effective, at dosages and for periods of time necessary, to achieve the desired therapeutic or prophylactic result.

Effective doses will be easily determined by one of skill in the art and will depend on the severity and course of the disease, the patient's health and response to treatment, the patient's age, weight, height, sex, previous medical history and the judgment of the treating physician.

The term “subject” means animals, such as dogs, cats, cows, horses, sheep, geese, and humans. Particularly preferred patients are mammals, including humans of both sexes.

The terms “treating”, “treat” and/or “treatment” include preventative (e.g., prophylactic) and palliative treatment.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Lactate is an Innate Activator of Mrs2-Mediated Mitochondrial Mg2+ Uptake

### A. Results

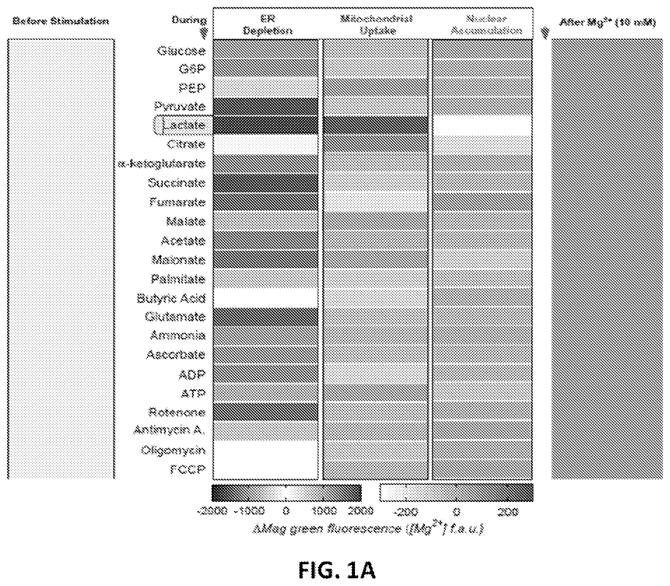

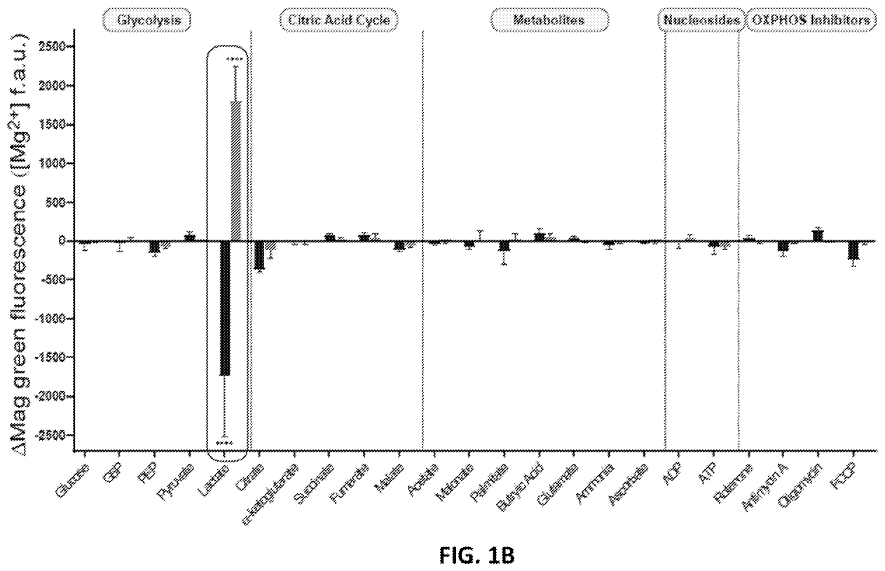

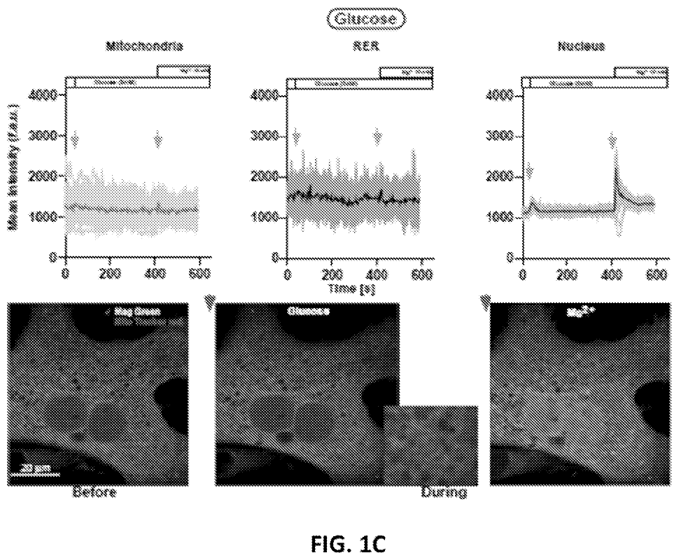

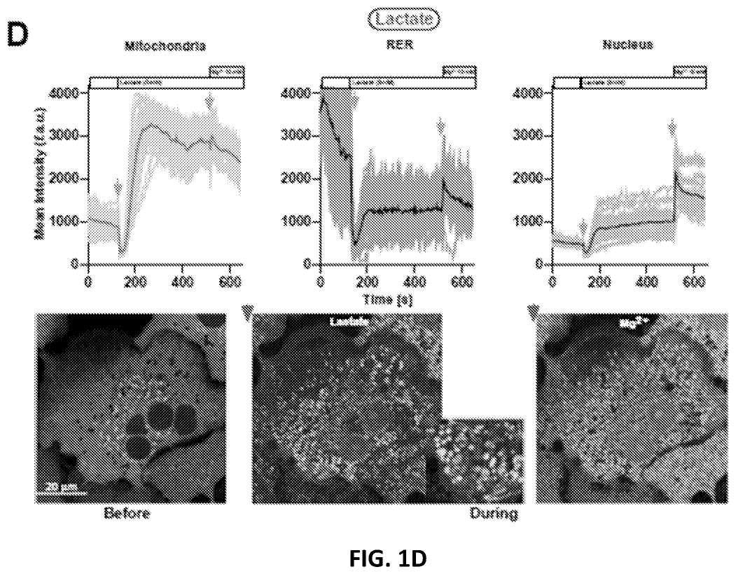

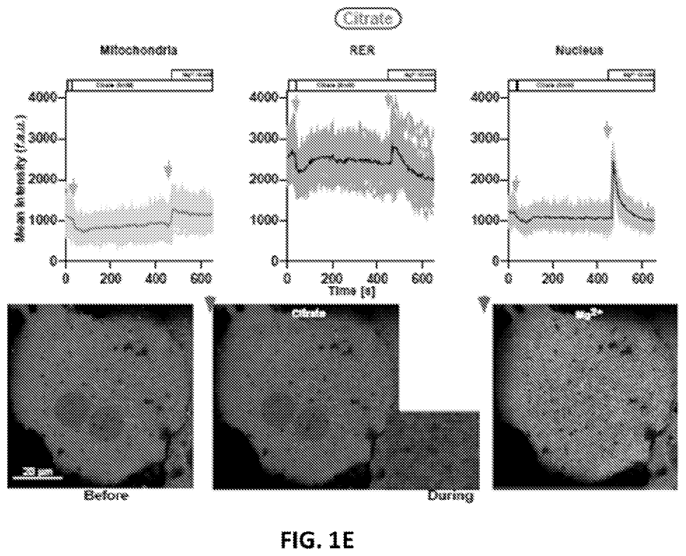

Targeted Metabolite Screen Identifies Lactate as activator agonist for Mitochondrial Mg²⁺ Uptake. To search for agonists that promote mitochondrial Mg²⁺ ([Mg²⁺]ₘ) uptake, the inventors developed a cell-based confocal fluorescence imaging. As an initial feature, the inventors tested whether GPCR agonist-induced second messengers promote Mg²⁺ dynamics. To discriminate intracellular Ca²⁺ and Mg²⁺ dynamics, MEFs were loaded with cell permeant Ca²⁺ indicator, Fluo-4-AM or Mg²⁺ indicator Mag green-AM followed by GPCR agonist, thrombin stimulation. As expected, application of thrombin rapidly elicited cytosolic Ca²⁺ mobilization, while intracellular Mg²⁺ dynamics were absent indicating that GPCR-derived second messengers like inositol-trisphosphate (IP₃) and diacyl glycerol (DAG) are not agonist/activators of [Mg²⁺]ᵢ dynamics. It is established that intracellular Ca²⁺ dynamics could be monitored using a wide range of fluorescent dyes or genetically encoded Ca²⁺ indicators. Conversely, Mg²⁺ sensitive indicators are very limited due to either lack of agonists or robust dynamic nature. To identify the agonist, primary murine hepatocytes were loaded with [Mg²⁺]ᵢ indicator, cell permeant Mag Green-AM (5 μM). Subsequently, dye loaded hepatocytes were stimulated with major cellular metabolites and mitochondrial oxidative phosphorylation (OXPHOS) inhibitors following baseline recording. Of the metabolites tested, aerobic/anerobic glycolytic end-product lactate consistently exhibited a rapid and robust depletion of ER Mag-Green signal followed by a subsequent elevation in the mitochondria (FIG. 1A). Notably, other products like pyruvate, glucose, palmitate, nucleotides, and other TCA intermediates did not induce mitochondrial Mg²⁺ uptake (FIG. 1A). As depicted in FIG. 1B, the reciprocal mobilization of free Mg²⁺ ion from ER to mitochondria was observed upon stimulation of Lactate (5 mM) in a temporal fashion. To confirm that lactate-induced Mag-Green signals are indeed measured from mitochondrial compartment, hepatocytes were simultaneously loaded with mitochondrial marker Mito-tracker Red and Mag-Green-AM (FIG. 1C-1F). To visualize the mitochondrial Mg²⁺ signal, hepatocytes applied to low (5 mM) or high glucose (25 mM) concentrations did not induce either ER depletion or enhance mitochondrial Mg²⁺ signal (FIG. 1C). Conversely, sodium lactate or lactic acid (5 mM) triggered a rapid depletion of ER signal with concomitant accumulation nucleus followed by mitochondrial elevation of Mag Green signal with a lag time of ˜25 s (FIG. 1D). Besides nucleotides such as ATP, citrate is also known to chelate free Mg²⁺. Since ATP is cell impermeable, citrate (5 mM) was applied and intracellular Mg²⁺ mobilization monitored. Interestingly, addition of citrate (5 mM) partly chelated both mitochondrial and ER Mg²⁺ signals suggesting its quenching effect (FIG. 1E). Once again, a robust uptake of Mg²⁺ into mitochondria was observed in three different cell types tested (mouse primary hepatocytes, HepG2 cell line, MEFs). To exclude the possibility that acidic pH of medium by lactate could alter membrane polarization, the pH was normalized. Additionally, the inventors also tested whether both sodium lactate as well as lactic acid induce mitochondrial Mg²⁺ uptake. Consistent with sodium form of lactate, lactic acid also elicited a rapid ER depletion followed by mitochondrial Mg²⁺ influx indicating a lactate role as an activator of Mg²⁺ dynamics. Thus, natural metabolite, lactate rapidly mobilizes ER Mg²⁺ which led to cytosolic elevation resulting in activation mitochondrial Mg²⁺ uptake.

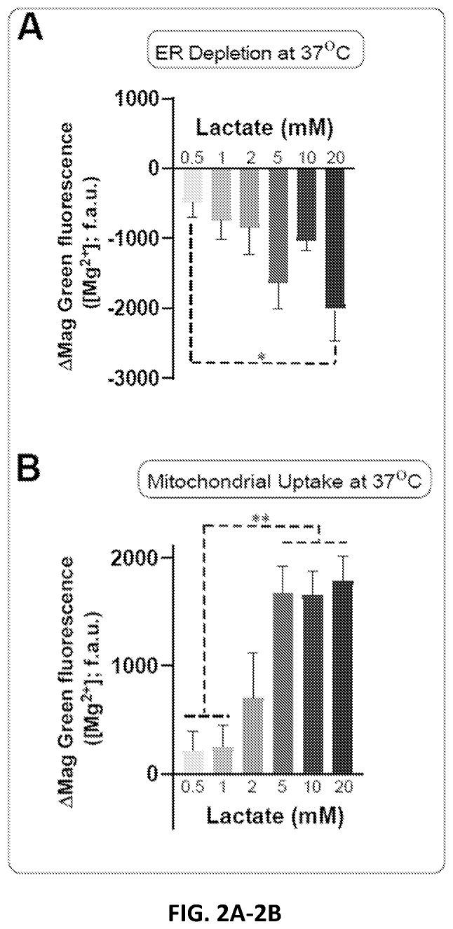

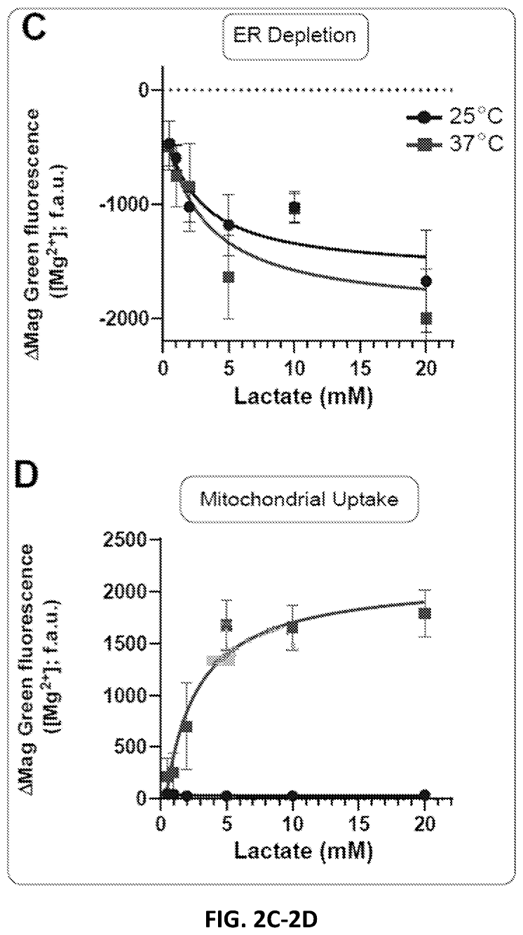

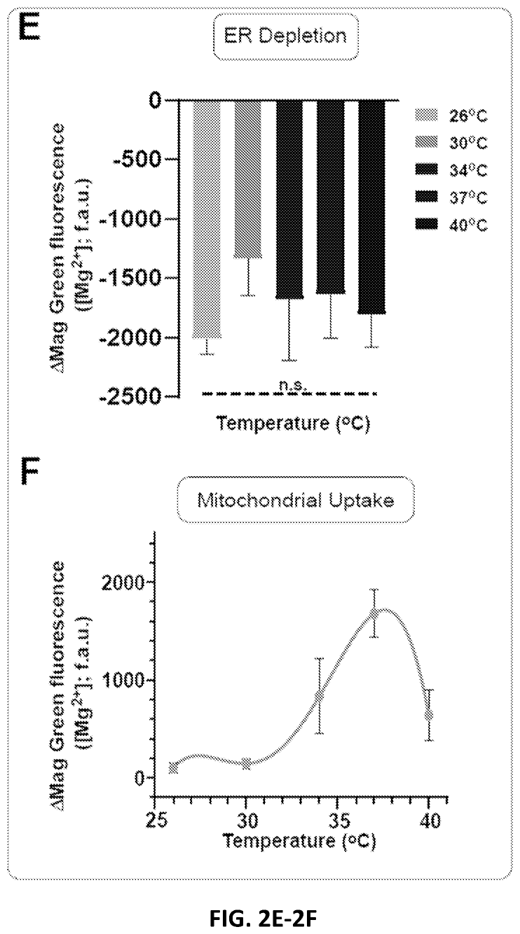

Mitochondrial Mg²⁺ Transporter is Temperature Sensitive. As revealed above, lactate is one of the carbohydrate catabolic end-products that accumulates normally in the range of ˜0.5-1.0 mM but higher levels (5.0-20.0 mM) were detected during intense exercise and several disease states. To assess optimal concentration of lactate required to trigger Mg²⁺ depletion from ER compartment and mitochondrial Mg²⁺ uptake, both ER-depletion and mitochondrial Mg²⁺ uptake was measured in hepatocytes at 37° C. The data clearly shows that lactate concentration≥2 mM promotes ER depletion and partial uptake of Mg²⁺ into mitochondria (FIG. 2A and 2B). However, a consistent stronger response was perceived at 5 mM lactate stimulation (FIG. 2A and 2B). During these studies depletion of ER Mg²⁺ was noticed at 37° C. and room temperature (25° C.) upon 5 mM lactate stimulation (FIG. 2C). Remarkably, mitochondrial Mg²⁺ uptake was higher at 37° C. and had no effect at 25° C. showing a temperature sensitivity (FIG. 2D). Based on this experimental evidence, a wide range of temperatures were tested. To determine the temperature responsiveness, primary hepatocytes were kept between 25° C.-40° C. and cells were stimulated with 5 mM lactate. Lactate-induced ER Mg²⁺ depletion was seen at all the indicated temperature range (FIG. 2E). However, lactate-induced maximal mitochondrial Mg²⁺ uptake was observed at 37° C. (FIG. 2F). Intriguingly, the lactate response was not seen at 25° C. or 30° C. (FIG. 2F). The lactate effect was sub-optimal at 34° C. and 40° C. indicating that mitochondrial Mg²⁺ uptake is temperature sensitive (FIG. 2F). Together, these results suggest that lactate-mediated mitochondrial Mg²⁺ uptake machinery is temperature sensitive.

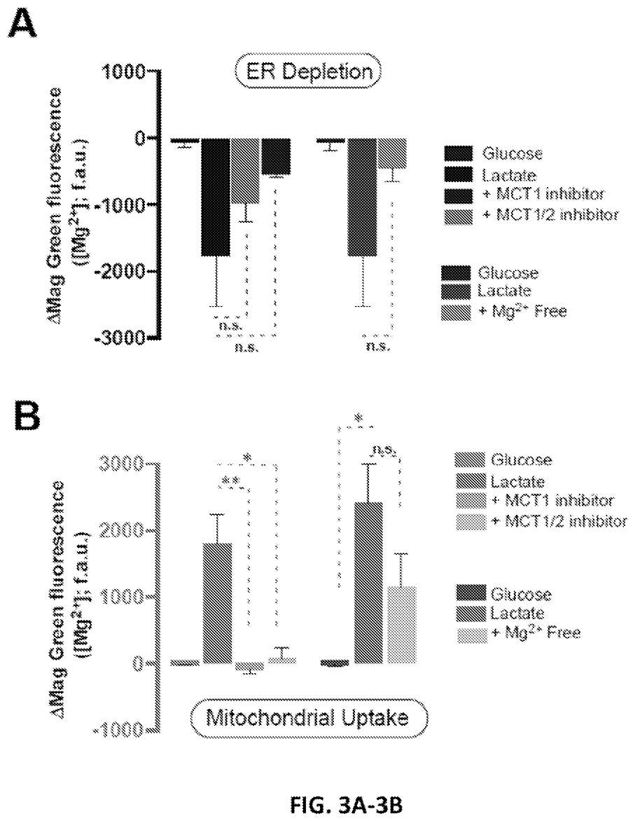

ER-Dependent Efflux of Free Mg²⁺ Promotes Mitochondrial Mg²⁺ Uptake. The results thus far suggest that lactate acts as a ligand/activator of mitochondrial Mg²⁺ uptake. Since extracellular delivery of lactate rapidly induces ER-depletion and subsequent mitochondrial uptake, it was asked whether rapid influx of lactate regulates this mechanism. Most mammalian cells utilize monocarboxylate transporters (MCT1-4) for plasma membrane lactate entry. To test this, primary hepatocytes were treated with MCT1 or MCT1/2 inhibitors for 60 minutes. Remarkably, both inhibitors abrogated lactate-induced Mg²⁺ uptake into mitochondria (FIG. 3A and 3B). However, it was observable that MCT1 inhibitor still showed partial ER-depletion but by blockade of MCT1 and 2 transporters significantly suppressed lactate effect suggesting that influx of lactate promotes ER-Mitochondrial Mg²⁺ dynamics (FIG. 3A).

Lactate-Induced Mitochondrial Mg²⁺ uptake Requires Mrs2. Having established that lactate elicits mitochondrial Mg²⁺ uptake, the inventors tested whether yeast Mrs2p homologue Mrs2 is the bonafide candidate for this process. A global Mrs2 knockout mouse model was generated by CRISPR/Cas9 gene editing strategy. Candidate sgRNAs were selected based on CRISPR/Cas9 nickase design platform, which substantially reduces off-target by ˜50-1500 fold. The selected paired gRNAs targeting exon 8 of Mrs2, the Cas9 nickase mRNA and single-stranded oligodeoxynucleotide (ssODN), for HDR-mediated gene KO were transfected in MEFs. Genotyping was performed by PCR-RFLP analysis with BamH1. The Mrs2p targeted allele was cleaved yielding two fragments (388 and 368 bp) whereas the wild type was not. After validation of gRNAs and ssODN in wild-type MEFs, two gRNAs, Cas9n mRNA and single-stranded oligodeoxynucleotide template (ssODN) were injected into C57BL6n zygotes and then implanted into pseudopregnant female mice. Founder mice were examined for ssODN incorporation by PCR amplification and restriction enzyme digestion (aa changes introduced a BamH1 cut site). After verifying germline transmission and breeding to heterozygosity DNA was isolated and sequenced to confirm the in-frame point mutations.

### Example 2

### Mrs2-Dependent Mg2+ Dynamics Purposes as Tunable Molecular Link for Whole Body Metabolism

### A. Results

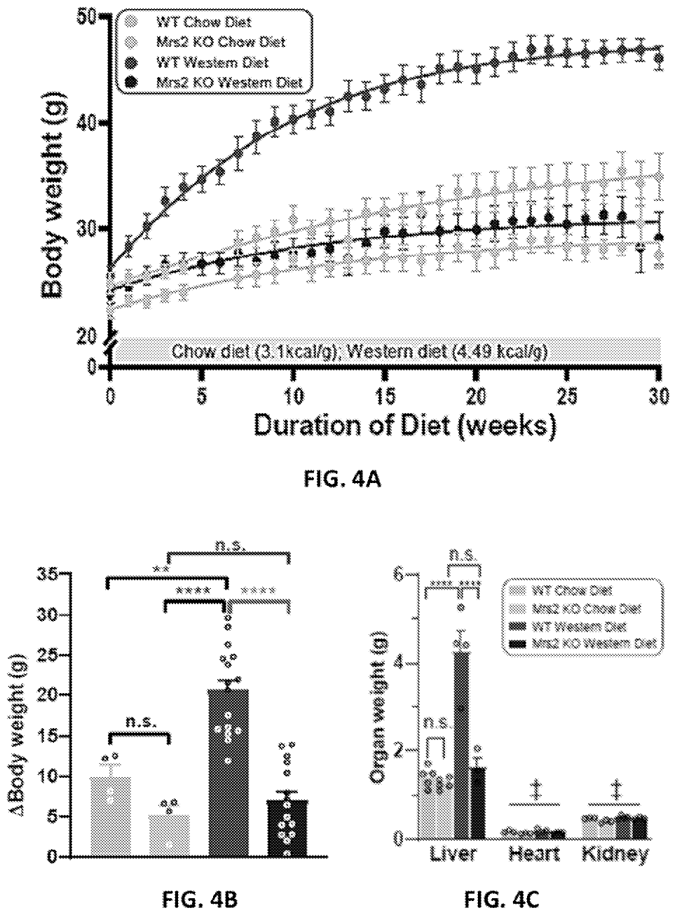

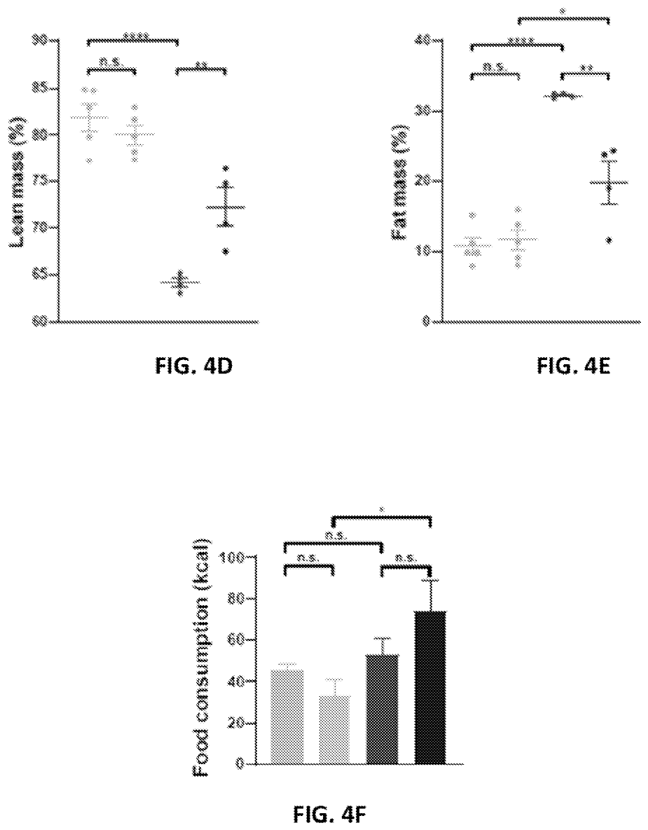

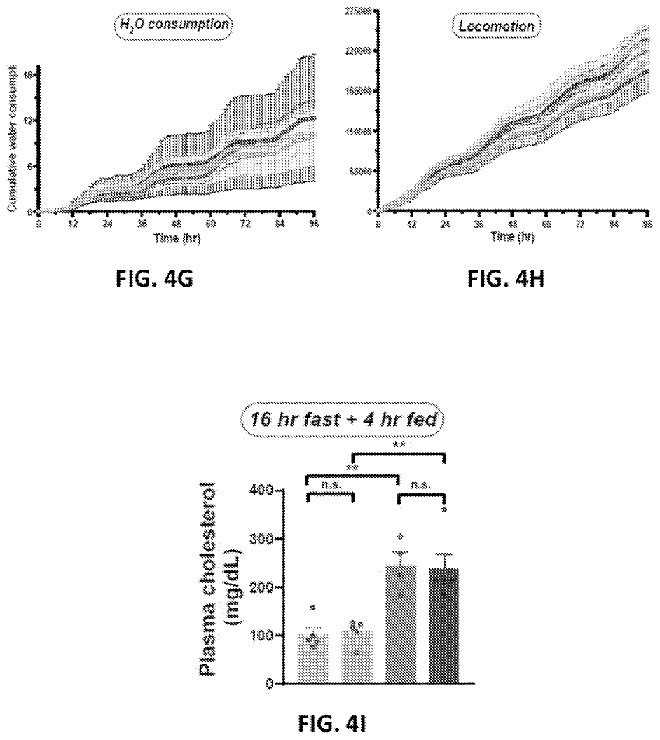

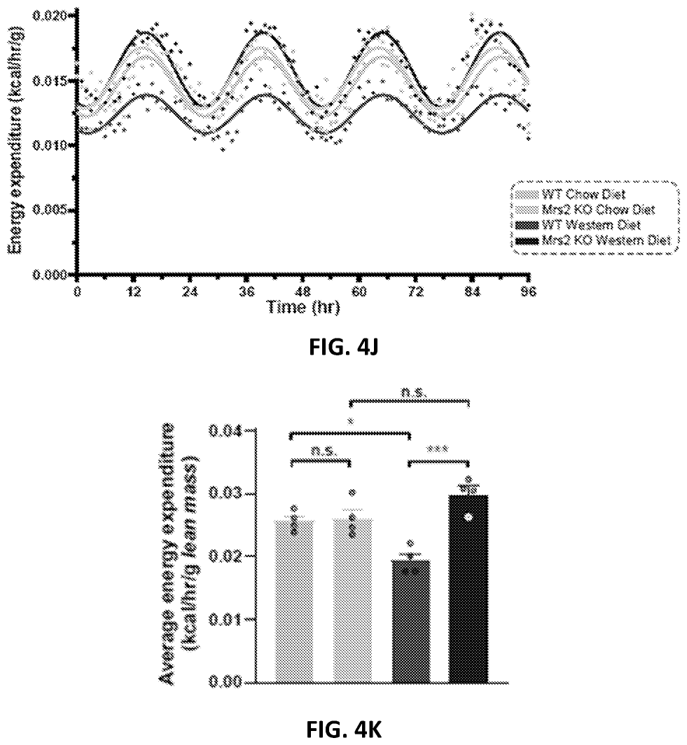

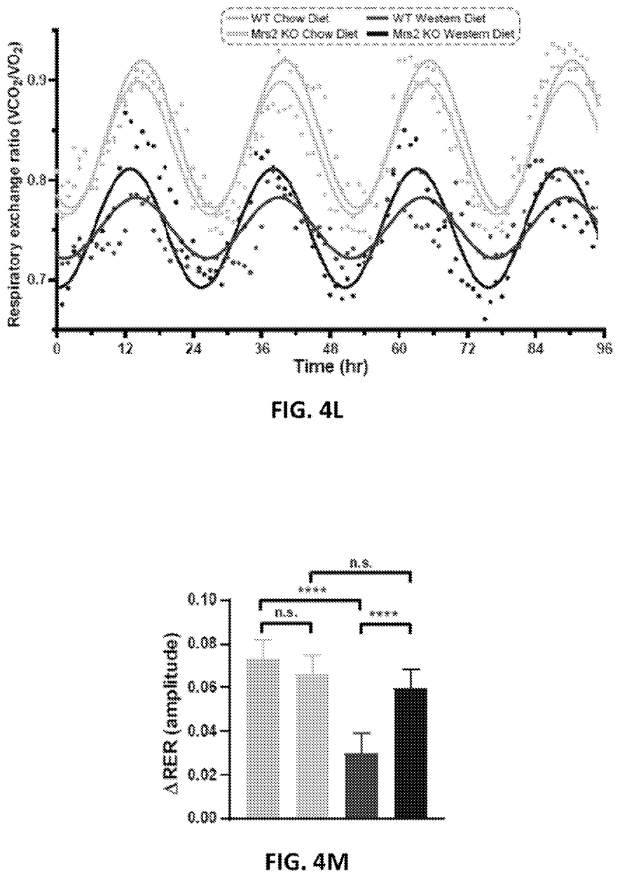

Lowering ₘMg²⁺ mitigates long-term Western diet-induced metabolic syndrome. The highly conserved bacterial CorA homologue, Mrs2, is an inner mitochondrial membrane-localized Mg²⁺ selective uptake machinery whose biological function is not well understood (Daw et al., 2020). To dissect the physiological significance of ₘMg²⁺ dynamics, a CRISPR/Ca9-mediated global Mrs2^{-/-} mouse model was established by introducing a premature stop codon to interrupt exon 8 (Daw et al., 2020). Using this model, it was recently demonstrated that limiting ₘMg²⁺ alleviates endotoxin-induced acute multi-organ failure via enhanced mitochondrial bioenergetics (Daw et al., 2020). Here, it was investigated whether long-term WD-induced metabolic syndrome could be prevented by perturbation of ₘMg²⁺ dynamics. 14 weeks old male mice were subjected to WD feeding for up to 52 weeks. The Western diet (WD) is composed of 40% (kcal) fat in comparison to 17% in the standard chow diet (CD) (Table 1). This protocol leads to obesity and deterioration of organ function. Wildtype (WT) and Mrs2 knockout (KO, Mrs2^{-/-}) mice were fed normal CD or obesogenic WD and body weights were regularly measured over 30 weeks. Remarkably, consumption of the Western diet caused obesity in WT but not Mrs2^{-/-} mice (FIG. 4A). The dietary protocol was continued up to 52 weeks before invasive studies. Even under this long-term dietary stress, loss of the mitochondrial Mg²⁺ uptake machinery (Mrs2) prevented severe weight gain associated with consumption of a Western diet (FIG. 4B). The characteristic hepatomegaly seen during obesity and NAFLD is observed in WD-fed WT mice but not in WD-fed Mrs2 KO mice; furthermore, the kidneys and heart remained normal size within each group indicating that liver and body weight differences were not due to changes in general growth (FIG. 4B, FIG. 4C). Body composition analysis by MRI mirrors the changes in fat and lean body mass with Western-diet feeding in WT or Mrs2^{-/-} mice. Intriguingly, the chow diet groups exhibited a normal fat/lean mass ratio highlighting a metabolic switch in the Mrs2^{-/-} mice upon consumption of an obesogenic diet. (FIG. 4D, FIG. 4E).

TABLE 1
Nutritional contents of various diet regimens.
CD, standard chow diet (Envigo 7012); WD,
Western diet (Research Diets D09100310).

| | Chow Diet | Western Diet |
|---|---|---|
| | (CD) | (WD) |
| Fat | 17% | 40% |
| Protein | 25% | 20% |
| Carbohydrate | 58% | 40% |
| Energy Content | 3.1 kcal/g | 4.49 kcal/g |

Mice were placed in indirect calorimetry chambers to examine energy expenditure and behavior. In contrast to WT mice, Mrs2^{-/-} mice fed WD maintained body weights close to those of chow fed controls (FIG. 4A FIG. 4B). This was not due to decreased food intake, water consumption, or locomotion, as WD-fed Mrs2^{-/-} mice ate as much as their WT counterparts (FIG. 4F, FIG. 4G, FIG. 4H). Mice were subjected to an overnight (16 hours) fast followed by ad libitum feeding for four hours with measurement of blood parameters. Since the Western diet contains 2% cholesterol it is expected that after eating, the WD-fed mice will have elevated plasma cholesterol. Indeed, plasma cholesterol was observed to be elevated in both WD-fed groups further demonstrating that eating habits and absorption are similar between the WT and Mrs2^{-/-} (FIG. 4I). Energy expenditure was observed to be lower in WT WD mice compared to Mrs2^{-/-} WD mice (FIG. 4J, FIG. 4K). No measurable difference in rectal body temperature was observed between the four groups of mice. Mice consuming WD were observed to have an overall decrease in respiratory exchange ratio (RER), as is expected with increased reliance on fat-based respiration (Patel et al., 2021). Consumption of WD and the associated metabolic syndrome additionally dampens oscillations of the RER between the light and dark cycles; however, loss of Mrs2 preserved oscillations in RER during dietary stress (FIG. 4L, FIG. 4M). Thus, lowering [Mg²⁺]ₘ by Mrs2 genetic ablation reveals a link between ᵢMg²⁺ dynamics and development of metabolic syndrome in response to WD.

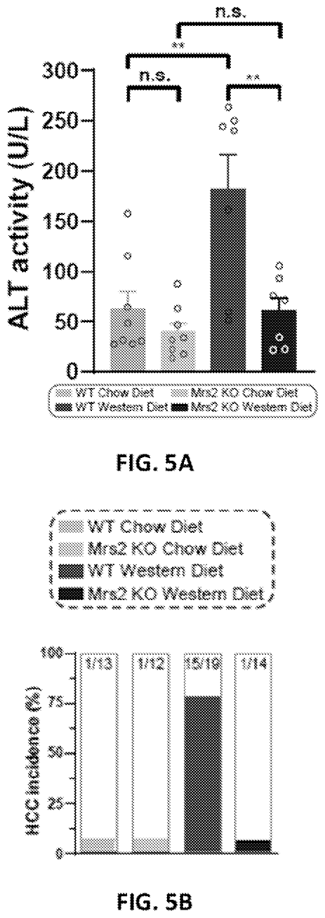

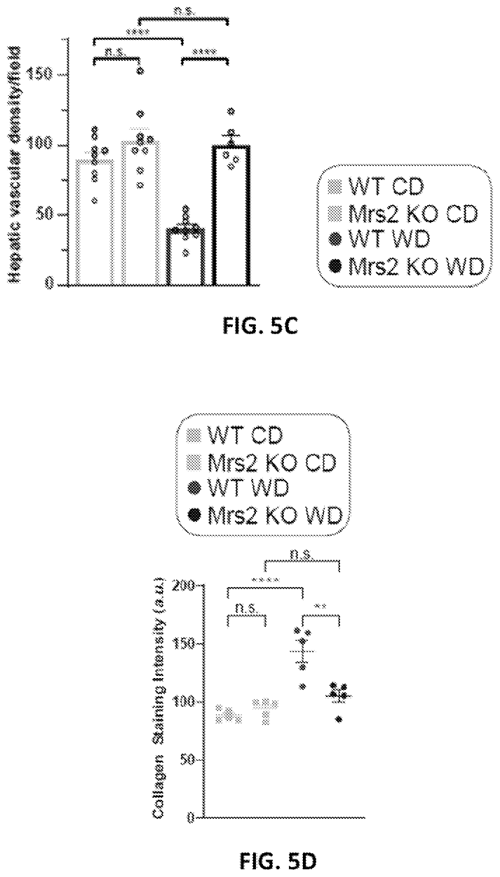

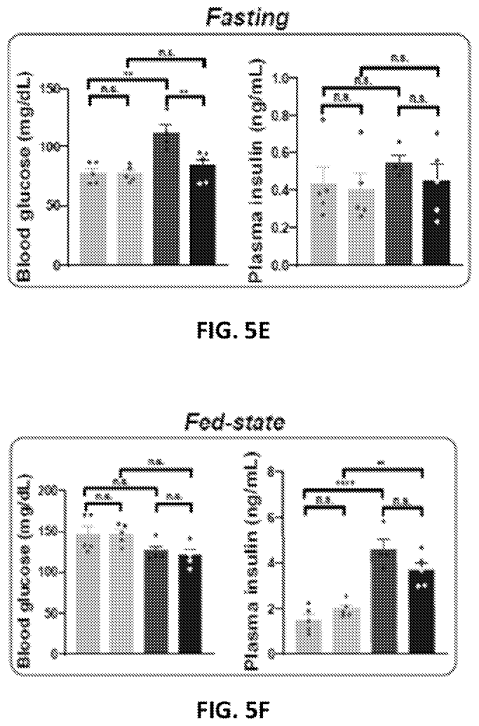

Deletion of Mrs2 prevents Western diet-induced liver steatosis and Microvascular rarefaction and spontaneous tumor prevalence. Having observed these gross anatomic and metabolic parameters, liver function was assessed by measuring the plasma levels of ALT (alanine transaminase), a marker of liver damage. Indeed, the WT WD mice, but not the Mrs2^{-/-} WD or the CD controls, had elevated ALT (FIG. 5A) (Reid, 2001). Consumption of a diet high in fat contributes to liver steatosis and eventual progression to hepatocellular carcinoma. Gross examination of the liver and mice revealed various stages of NAFLD in WT WD mice, but remarkably, no liver disease state was observed in the Mrs2^{-/-} WD mice. Fatty deposition, neoplastic lesions, and enlargement are all characteristic of NAFLD and could be seen in liver of obese mice (WT WD) but not Mrs2^{-/-} mice. Microscopic examination of the livers from Western diet-fed mice revealed steatosis in WT WD but not in Mrs2^{-/-} WD or CD controls. WT WD-fed mice had increased extra-hepatic and intra-hepatic lipid deposition in addition to increased collagen formation. Mrs2^{-/-} WD mice presented with no neoplastic lesions compared to WT WD mice and tumor incidence after 1 year of diet was similar to chow diet fed mice. (FIG. 5B) Microvascular rarefaction (microvascular reduction) is a phenomenon that occurs commonly during aging-associated cardiovascular diseases (Ungvari et al., 2021). To investigate microvascular densities in both CD and WD diet groups, liver sections were immunostained with the endothelial marker CD31/PECAM1 (Grunewald et al., 2021). CD31⁺ staining revealed decreased capillary density in WT obese mice (FIG. 5C). In contrast, WD failed to cause microvascular rarefaction in Mrs2^{-/-} mice (FIG. 5C). Closer examination using electron microscopy revealed numerous large lipid droplets in WT WD-fed mice that were absent in Mrs2^{-/-} WD hepatocytes; additionally, large amounts of glycogen granules were observed in the WD-fed Mrs2^{-/-} but not in the WD WT liver. Since WD-induced obesity is associated with chronic kidney disease, the cortex was evaluated for fibrosis by examining collagen deposition. Masson's trichrome staining of coronal sections showed collagen accumulation in WD-induced WT mice, while collagen deposition was nearly absent in WD-fed Mrs2^{-/-} mice (FIG. 5D). As typically seen in obese mice, the fasting blood glucose levels were higher in WT WD, but the Mrs2^{-/-} mice exhibited normal glucose that was comparable to control diet (FIG. 5E). Fasting insulin levels were relatively similar between the groups but trended higher in the obese mice (FIG. 5E). Although refed glucose levels were similar between the four groups, they were slightly elevated in the CD group (FIG. 5F), whereas post-prandial insulin levels were increased in the WD groups (FIG. 5F). Together, these data demonstrate that controlling [Mg²⁺]ₘ prevents changes associated with diet-induced metabolic syndrome.

HIF-1α pathway as a switchable link in the Western-diet induced obesity. Although lowering [Mg²⁺]ₘ enhances the mitochondrial oxidative decarboxylation cascade and OXPHOS activity, the physiological and molecular phenotypes of Mrs2^{-/-} mice have never been described in detail (Daw et al., 2020). To identify the causal link between ᵢMg²⁺ dynamics and metabolic disease progression, we performed global RNA profiling of liver and adipose tissues were performed. The panoramic transcriptomic changes on liver from WT WD and Mrs2^{-/-} WD showed that ≈5500 protein-coding genes were differentially regulated, of which more than 200 are involved in metabolic diseases and cellular processes such as glucose and fatty acid metabolism and mitochondrial bioenergetics, consistent with the antagonistic role of Mg²⁺ against Ca²⁺ dependent signaling (Daw et al., 2020; Ramachandran et al., 2022). However, comparison of livers from WT and Mrs2^{-/-} CD revealed that only nine genes were significantly changed and seven were differentially regulated in both dietary conditions. WT WD liver samples exhibited a significant elevation of markers of tumorigenesis, inflammation, and fibrosis when compared with Mrs2^{-/-} WD (FIG. 6A, FIG. 6B) (Kanehisa, 2019; Kanehisa et al., 2021; Kanehisa and Goto, 2000). Thus, loss of Mrs2 prevented severe metabolic-like syndrome and the emergence of diet-induced NAFLD and hepatocellular carcinoma in WD fed mice. Unbiased transcription factor analysis using ChEA software demonstrated HIF1-α as a likely candidate responsible for the transcriptional program seen in the livers of WD-fed animals. Remarkably, HIF1-α dependent signaling was observed to be differentially regulated in WD-fed mice. Furthermore, KEGG based analysis of thermogenesis, oxidative phosphorylation, fatty acid catabolismglycolysis, TCA cycle, and the hypoxia-inducible pathway revealed substantial metabolic pre-programming in Mrs2^{-/-} liver that afforded a pro-catabolic state in the liver tissue upon chronic Western diet consumption. (Kanehisa, 2019; Kanehisa et al., 2021; Kanehisa and Goto, 2000). As mitochondrial Ca²⁺ is a key driver for bioenergetics, whether long-term WD-induced suppression of MCU alters OXPHOS protein complex abundance was determined. Consistent with RNAseq data sets, the WD regimen exhibited suppression of Tom20 and OXPHOS complex abundance, suggesting loss of mitochondrial density that was rescued in Mrs2^{-/-} WD.

Changes in the transcriptional profiles of inguinal white adipose tissue (iWAT) where assessed. Although gene expression profiles were changed considerably in this tissue, including 74 protein coding genes differentially regulated in both dietary conditions with all of them regulated in the same direction, again the focus was on the major metabolic pathways and HIF1α signaling (Kanehisa, 2019; Kanehisa et al., 2021; Kanehisa and Goto, 2000). Using the ChEA unbiased transcription factor analysis software on iWAT differenentially regulated genes revealed HIF1-α as a strong candidate for controlling the transcriptional program in the inguinal WAT of livers of WD-fed animals. Since a massive elevation of thermogenic candidates was observed under CD as well as WD in the iWAT of Mrs2^{-/-} mice, histologic analysis was performed which revealed a browning of iWAT tissue in Mrs2^{-/-} CD that was amplified when fed with WD in Mrs2^{-/-} as compared the respective WT groups. Next the vascular density in the adipose tissue was measured and was found to be significantly increased in Mrs2^{-/-} WD. Specific markers for brown/beige adipocytes were quantified. As expected, the mt-encoded OXPHOS and ATP synthase subunits were significantly elevated in Mrs2^{-/-} WD vs WT WD. Similarly, the RNA-seq analyses showed upregulation of beige markers and fatty acid catabolismgenes in Mrs2^{-/-} WD mice. Quantification of (Jepl and Lep gene expression in iWAT tissue by RT-qPCR mirrored the RNA-Seq findings. Upon examination of the glycolytic pathway, HIF-1α controlled glycolytic transcripts were significantly upregulated in Mrs2^{-/-} WD mice. How mitochondrial function was changed in the iWAT of Mrs2^{-/-} animals was examined using fresh fat explants and homogenate. Spectacularly, the phenotype of TMRE mitochondrial staining mirrored the RNA-Seq findings. Western diet-fed WT animals had hypertrophic, unilocular adipocytes with minimal mitochondrial network; meanwhile, Mrs2^{-/-} WD had smaller, multilocular, adipocytes with a heavy mitochondrial network (FIG. 6C). The fat homogenate of Mrs2^{-/-} animals had improved bioenergetics compared to WT animals. Overall, the RNA-seq data further highlight the known HIF-1α driven regulation of pyruvate transport, OXPHOS complex, and other mitochondrial nuclear-encoded transcripts (Bishop and Ratcliffe, 2020; Iliopoulos et al., 1996;Semenza, 2014; Yang et al., 2014). Comparing the transcriptional data with Mg²⁺ binding proteins revealed substantial overlap (UniProt, 2021), suggesting the potential for direct regulation and adaptive mechanisms in the absence of Mrs2 channel.

Western diet induced suppression of mitochondrial fatty acid flux and Ca²⁺ uptake machinery is prevented in Mrs2 KO mice. To understand the molecular changes that facilitate glucose and lipid oxidation in Mrs2^{-/-} mice in response to WD, hepatocytes were isolated from WT and Mrs2^{-/-} mice fed control or WD for 52-54 weeks. Overnight cultured hepatocytes were co-stained with the lipid indicator BODIPY and the mitochondrial potential indicator TMRE to quantify lipid droplets and mitochondrial ΔΨₘ using confocal microscopy. The WT WD hepatocytes exhibited more and larger lipid droplets when compared to other groups (FIG. 7A, FIG. 7B). It is noteworthy that hepatocytes isolated from WT WD mice often failed to adhere on the collagen, gelatin, poly-lysine, or fibronectin-coated dishes, hinting a massive intracellular lipid accumulation and higher tendency to float in the culture medium. A closer examination of the lipid droplets and mitochondria revealed a preservation of lipid droplet-mitochondria homeostasis in Mrs2^{-/-} WD, but not in WT WD. Specifically, lipid droplets occupied less than 10% of the extra-nuclear region in CD groups and Mrs2^{-/-} WD hepatocytes but this was increased to approximately 20% in the WT WD hepatocytes. (FIG. 7B). Upon visualization of hepatocytes, a unique mitochondrial BODIPY signal phenotype was observed that occurs in Mrs2^{-/-} subjected to WD. Colocalization analysis of mitochondria further confirmed mitochondrial localization of BODIPY in the Mrs2^{-/-} WD group (FIG. 7C). Having observed mitochondrial BODIPY accumulation, the abundance of the mitochondrial fatty acid transporter carnitine palmitoyltransferase-1A, a mitochondrial outer membrane integral protein, was assessed in liver tissue harvested from all four groups. Remarkably, the CPT1A protein abundance was suppressed in WT WD, but its level was substantially maintained in Mrs2^{-/-} livers, suggesting the higher potential for fatty acid β-oxidation to fuel bioenergetics (FIG. 7D upper panel).

Since MCU-mediated mitochondrial Ca₂₊ flux is essential for numerous metabolic oxidative enzymes including glucose and fatty acid oxidation, TCA cycle, and electron transport chain complex activities, liver tissue lysates were subjected to Western analysis to determine the MCU and MICU1 protein abundance (Alevriadou et al., 2021). WT WD showed a striking low abundance of MCU and MICU1 while Mrs2^{-/-} WD MCU complex abundance was comparable to WT or Mrs2^{-/-} CD mice (FIG. 7D lower panel). To further establish that MCU-mediated Ca²⁺ uptake and mitochondrial Ca²⁺ handling is necessary for proper bioenergetics, MCU complex activity and its major driving force ΔΨₘ were measured in hepatocytes isolated from these four groups. Using a permeabilized hepatocyte model, it was found that the MCU-mediated Ca²⁺ uptake and matrix Ca²⁺ retention capacity were strikingly increased in Mrs2^{-/-} CD when compared to WT CD hepatocyte mitochondria (FIG. 7E, FIG. 7F). Additionally, it was found that WT WD hepatocyte's mitochondria exhibited complete abrogation of MCU-dependent Ca²⁺ uptake but Mrs2^{-/-} WD hepatocytes significantly retained these activities, being comparable to hepatocytes from WT CD mice. Despite the impaired handling of Ca²⁺ and fatty acids, ΔΨₘ was apparently not compromised in WT WD hepatocytes (FIG. 7G). Because ₘCa²⁺ controls bioenergetics, and alterations of MCU activity in WT WD may account for the differences observed in the extra-hepatic and intracellular lipid accumulation, it was hypothesized that the mitochondrial oxygen consumption rate (OCR) had succumbed in this group. Overnight cultured hepatocytes from these cohorts were tested for basal respiration, maximal O₂ consumption and mitochondrial proton uncoupling using a Seahorse flux analyzer. Basal, uncoupler-induced maximal, and proton leak were significantly suppressed in WT WD hepatocytes as compared to the other three groups (FIG. 7H, FIG. 7I). Additionally, the extracellular acidification rate, an index of glycolysis, was partially maintained in Mrs2^{-/-} WD as compared to WT WD hepatocytes. These results indicate that WD induced lipid accumulation in the liver could be due in part to decreased mitochondrial fatty acid utilization, consequent remodeling of hepatic nuclear encoded transcripts, and protein complex activities that are ᵢMg²⁺ dependent.

Efflux of the de novo lipogenesis precursor and endogenous Mg²⁺ chelator citrate is blunted in Mrs2 KO mice. Production of acetyl-CoA from citrate is a key node in the synthesis of cholesterol and fatty acids, that contribute to hyperlipidemia and deposition of triglyceride species in tissues (Pinkosky et al., 2017). Because citrate is the major precursor of de novo lipogenesis in metazoans, the plasma citrate levels were measured from these four cohorts. Blood samples were drawn from these mice before harvesting organs for RNA-Seq and histological studies. As depicted the plasma citrate concentration was not significantly affected by WD in WT mice (FIG. 8A). However, the plasma citrate was considerably lower in Mrs2^{-/-} mice, regardless of diet (FIG. 8A). The differences in plasma citrate prompted the examination of mitochondrial citrate efflux, since the TCA cycle is the major source. To examine the mitochondrial citrate [mcitrate] concentration ([ₘcitrate]), the newly created mitochondria-targeted genetically encoded citrate sensor mitoCitron was transiently constituted in HepG2 cells (Zhao et al., 2020). Upon stimulation with high glucose (17 mM), the mitoCitron signal was rapidly increased, followed by a dissipation that corresponds to citrate production and subsequent utilization or efflux from the mitochondria (FIG. 8B). Next both cytosolic and mitochondria citrate sensors were expressed in WT and Mrs2^{-/-} hepatocytes. Mitochondrial citrate production was higher in Mrs2^{-/-} hepatocyte mitochondria; however, the appearance of cytosolic citrate in Mrs2^{-/-} hepatocytes was greatly reduced following glucose stimulation (FIG. 8C, FIG. 8D. Additionally, the loss-of-function Mrs2 mutant was reconstituted in WT hepatocytes to measure the cytosolic and mitochondrial citrate concentration. Consistent with Mrs2^{-/-} , the Mrs2 mutant expressing hepatocytes exhibited lower cytosolic citrate flux when compared to control (FIG. 8E, FIG. 8F). To confirm these differences in citrate dynamics as not due to changes in Slc25a1 (mitochondrial citrate carrier) expression, hepatocytes from WT and Mrs2^{-/-} mice (both CD and WD fed animals) were probed for Slc25a1 expression. Slc25a1 expression was observed to be similar in cells from CD-fed animals and slightly reduced in WD-fed conditions. (FIG. 8G) RNA-seq analysis of liver tissue from these animals correlated perfectly with the cellular data. The decreased citrate efflux from Mrs2^{-/-} mitochondria is not attributed to reduced Slc25a1 expression as observed by similar levels of gene expression; this data suggests that ₘMg²⁺ could regulate SLC25a1-mediated citrate efflux. Furthermore, the citrate dynamics in the HepG2 cell line were evaluated, and the data are consistent with murine hepatocytes (FIG. 8H, FIG. 8I).

A fluorometric assay was developed to determine the extramitochondrial citrate accumulation following a supplementation of citrate precursor oxaloacetate. Citrate is the second strongest endogenous chelator of ᵢMg²⁺ (London, 1991), a property exploited by utilizing the Mg²⁺ fluorescent indicator Mag-Green (K_{D}˜1 mM) (Daw et al., 2020). Because the binding affinity for citrate and Mg²⁺ complex (K_{D}˜0.48 mM) (London, 1991) is superior to MagGreen's Mg²⁺ binding affinity, the rate of Mag-Green intensity loss (quenching) is a function of citrate accumulation in the cytosol. WT or Mrs2^{-/-} hepatocytes were permeabilized and bathed with 5 μM Mag-Green before 1 mM MgCl₂ bolus delivery. The extramitochondrial Mg²⁺ was rapidly taken up by WT but not Mrs2^{-/-} hepatocyte mitochondria (FIG. 8J) (Daw et al., 2020). After steady state (200 s), 5 mM oxaloacetate was added in the cuvette and the reduction of Mag-Green fluorescence was monitored as a readout of citrate efflux from the mitochondria. The cytosolic accumulation of citrate was observed in WT hepatocytes, but Mrs2^{-/-} hepatocytes did not show any signal of citrate release. Thus, Mrs2^{-/-} hepatocytes have reduced citrate efflux from mitochondria.

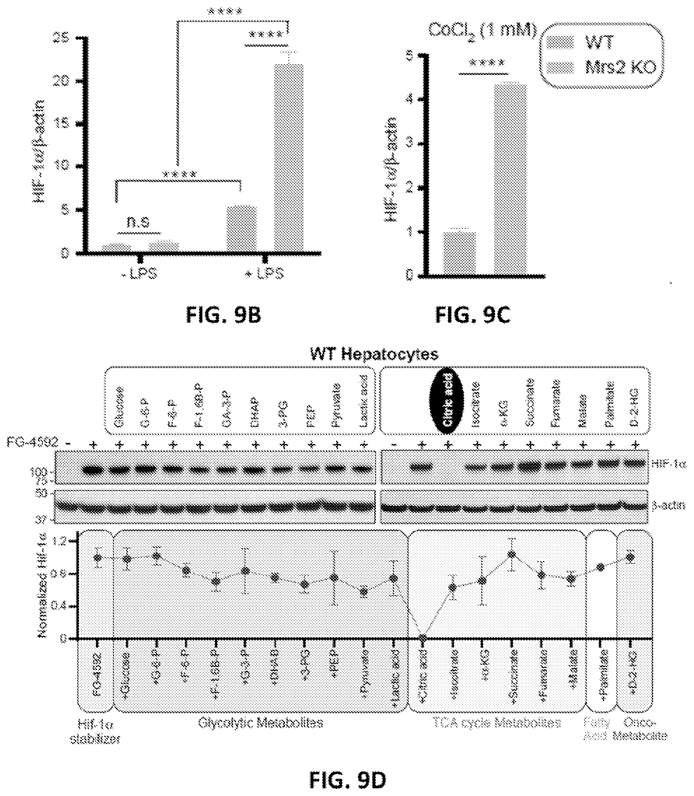

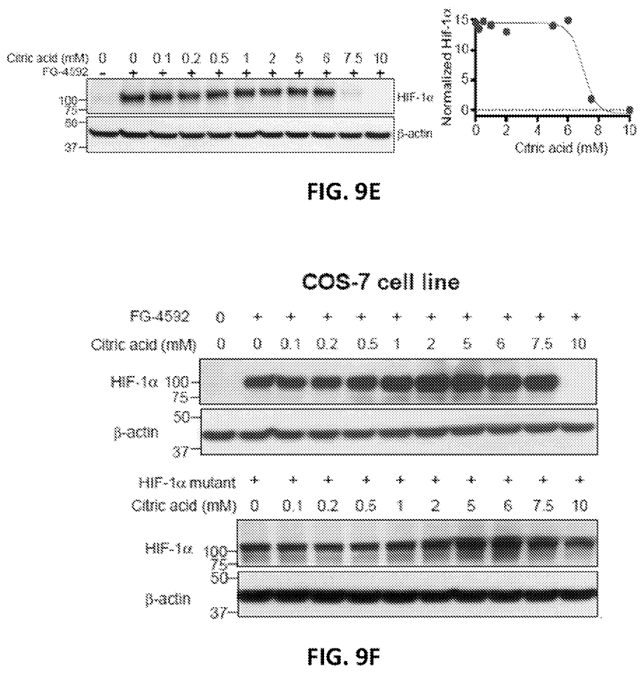

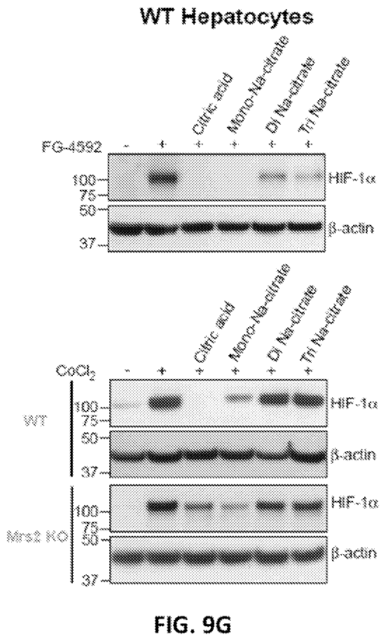

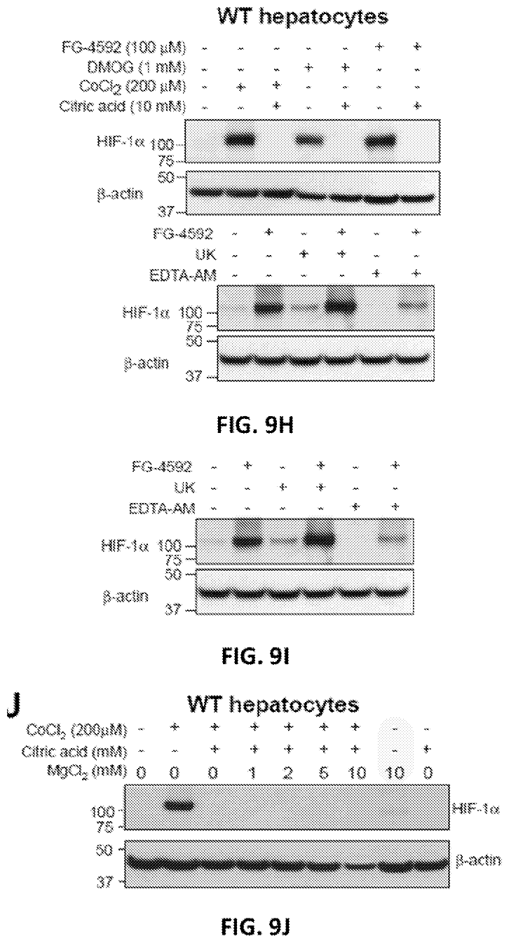

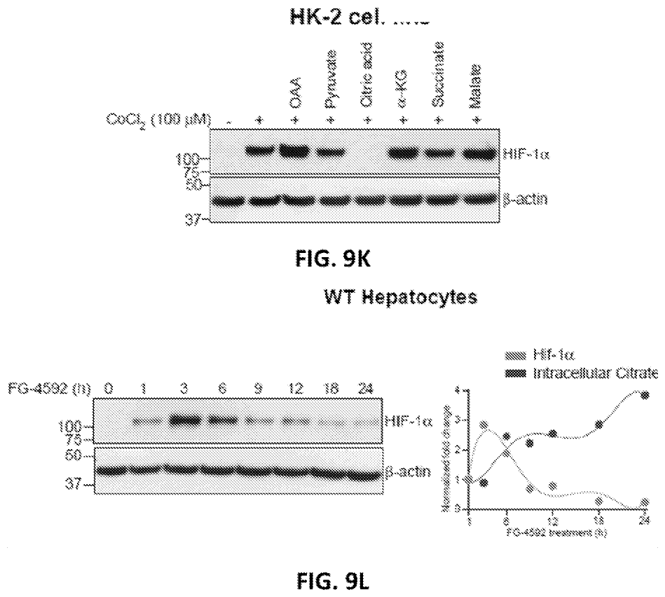

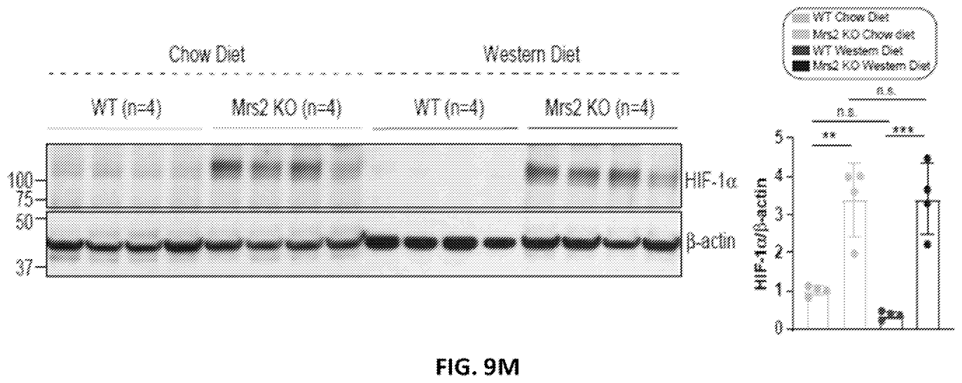

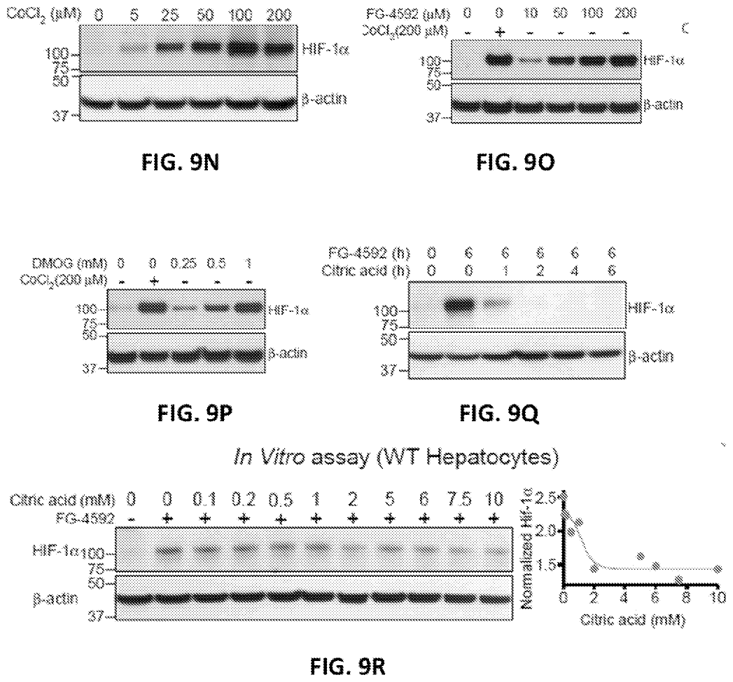

HIF-1α is stabilized in Mrs2−/− mice. To examine the intrinsic role of Mrs2, Mrs2^{-/-} mice were challenged to LPS-induced inflammatory response. It was noticed that there was a barely detectable HIF-1α stabilization in control but a significant HIF-1α protein accumulation following LPS stimulation in Mrs2^{-/-} hepatocytes (Daw et al., 2020). Having observed the remarkable response against an inflammatory stimulus, the HIF-1α stabilization was compared between genotypes following stimulation with LPS or the generic prolyl hydroxylase (PHD) inhibitor cobalt chloride (CoCl₂). Hepatocytes derived from Mrs2^{-/-} mice showed higher accumulation of HIF-1α (FIG. 9A, FIG. 9B, FIG. 9C). It was envisioned that loss of Mrs2 in hepatocyte mitochondria might control metabolite fluxes that regulates HIF-1α stability under normoxic conditions. HIF-1α is the most commonly studied transcription factor that responds to oxygen tension and its stabilization controls numerous metabolic cascades, including glycolysis and oxidative phosphorylation (Kaelin and Ratcliffe, 2008; Lum et al., 2007; Semenza, 2013). A targeted metabolite screen was conducted to identify whether these metabolites potentially altered in Mrs2^{-/-} control HIF-1α stabilization. WT hepatocytes were treated with the PHD inhibitor FG-4952 for 6 hours in the presence or absence of metabolites (Buckley et al., 2012). Of the conditions tested, the presence of citric acid destabilized HIF-1α (FIG. 9D) and its effect was observed at a concentration≥6 mM (FIG. 9E). Next it was explored whether citric acid mediated HIF-1α destabilization is a hydroxylation dependent mechanism by reconstitution of hydroxylation-dead mutant HIF-1α in COS-7 cells (Yan et al., 2007). As expected, 10 mM citric acid completely blunted FG-4592 dependent stabilization (FIG. 9F top panel). In contrast, constitutively stabilized HIF-1α mutant was unaffected by the presence of citric acid suggesting that the hydroxylated HIF-1α is the target for this mechanism (FIG. 9F bottom panel). Next tested the potency of citric acid and sodium citrate was tested. Hepatocytes isolated from WT or Mrs2^{-/-} mice were treated for 6 hours. HIF-1α stabilization was less affected by di- or tri-sodium citrate than by citric acid (FIG. 9G). This suggests that pH plays a role in the destabilization of HIF-1α and may be permissive for any effect of citrate per se. Notably, hepatocytes from Mrs2^{-/-} mice were partially resistant to the effect of 10 mM citric acid treatment suggesting the possibility that they more effectively buffer pH, have lower intracellular citrate concentration, or have higher [Mg²⁺]_{c} that sequesters exogenous citrate (FIG. 9G bottom pane). The citric acid-mediated HIF-1α destabilization was intact in multiple chemical modalities (FIG. 9H) and the intracellular chelation of Mg²⁺ ions suppressed FG-4952-induced HIF-1α stabilization (FIG. 9I). Furthermore, it was shown that blockade of mitochondrial pyruvate entry with UK5099 (UK) did not prevent HIF-1α stabilization (FIG. 9I). The effect of higher levels of Mg²⁺ on citrate-mediated HIF-1α destabilization were evaluated and found it to be weakly ᵢMg²⁺ dependent; high levels of Mg²⁺(10 mM) were sufficient to promote low levels of HIF-1α stabilization (FIG. 9J). To determine whether the citrate precursor oxaloacetate (OAA) or citrate product α-ketoglutarate (α-KG) exert similar functions, first human kidney cell line (HK-2) treated with CoCl₂ were tested in the presence or absence of metabolites for 6 hours and measured HIF-1α stabilization. Citric acid treatment replicates the observations in hepatocytes; however, OAA, α-KG, and others do not possess such activity (FIG. 9K). In hepatocytes, CoCl₂, FG-4592, and DMOG all promoted HIF-α stabilization, with higher concentrations causing increased stabilization (FIG. 9N, FIG. 9M, FIG. 9P). Citric acid-induced HIF-1α destabilization was very rapid and robust (FIG. 9Q). The citric acid effect was partly maintained in the FG-4592 treated WT hepatocyte homogenate (FIG. 9R). Furthermore, COS-7 cells were more sensitive to the HIF-1α stabilizing effect compared to hepatocytes. It was found that HIF-1+ abundance decreased over time in hepatocytes treated with FG-4592 (100 μM) and correlates with citrate accumulation but is not clearly related to pH changes in hepatocytes. Next HIF-1α abundance was tested in liver tissues harvested from dietary cohorts. HIF-1α protein expression/stabilization was prominent in both Mrs2^{-/-} and Mrs2^{-/-} WD, whereas HIF-1α protein was nearly completely absent in WT WD (FIG. 9M). This supports the elevation of HIF-1α protein level as a key mechanism driving transcription changes in Mrs2^{-/-} WD livers.

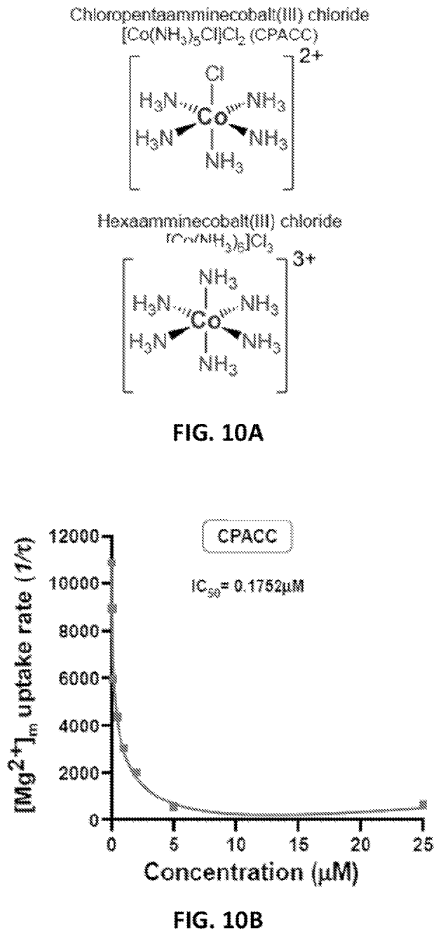

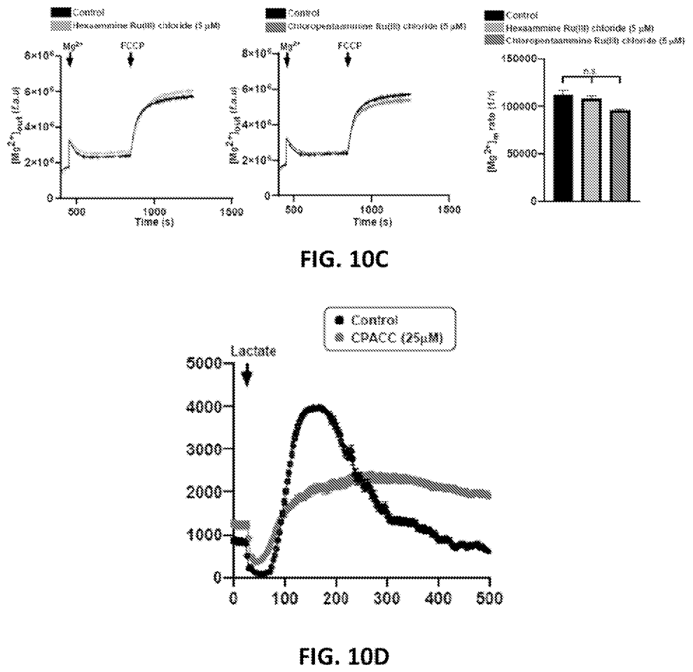

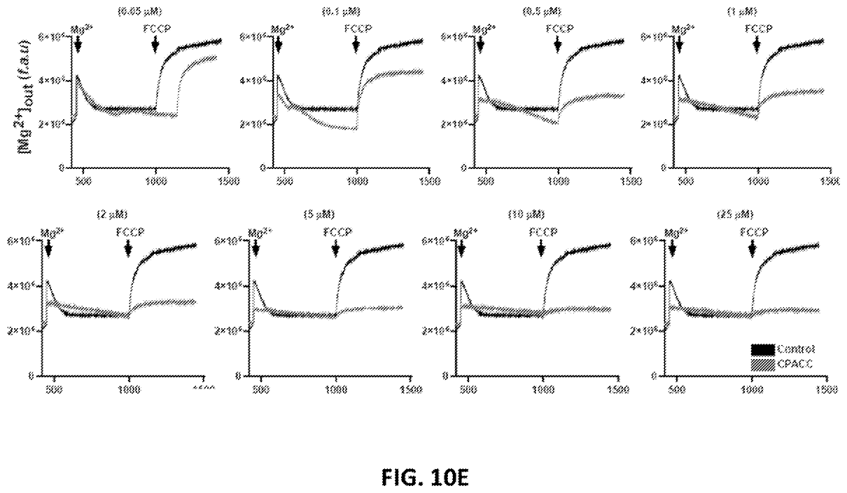

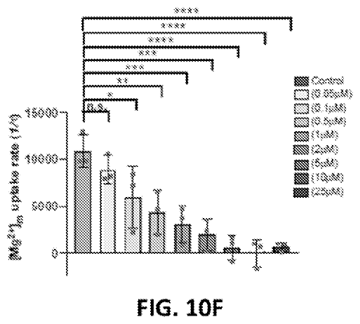

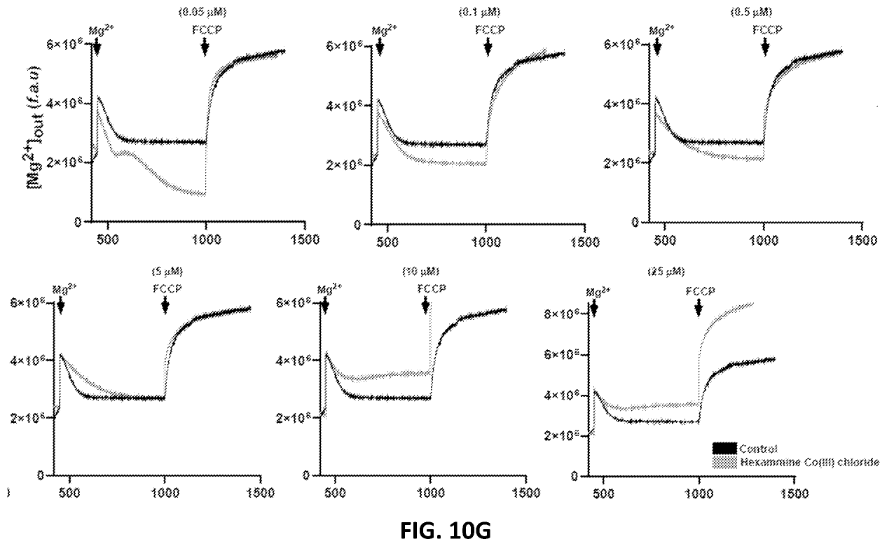

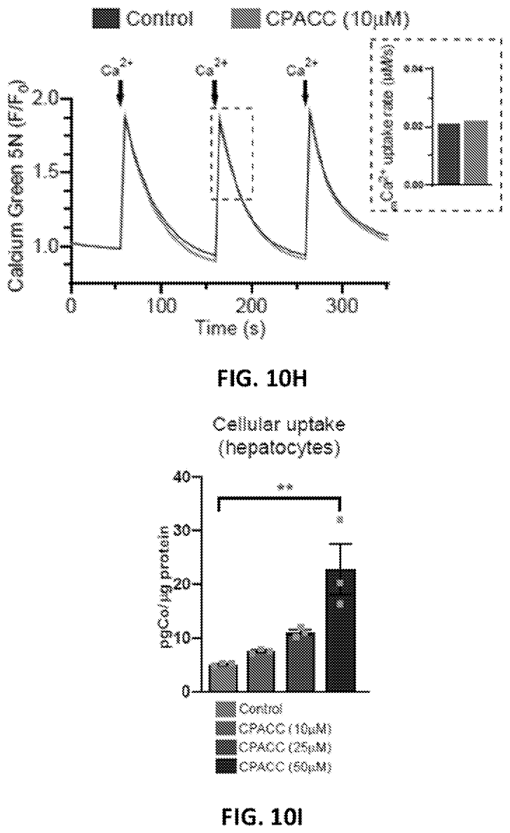

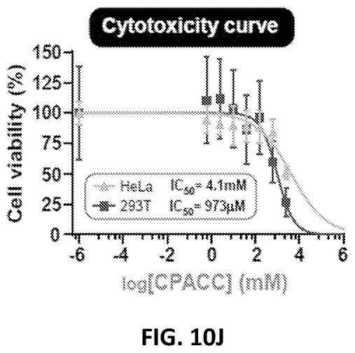

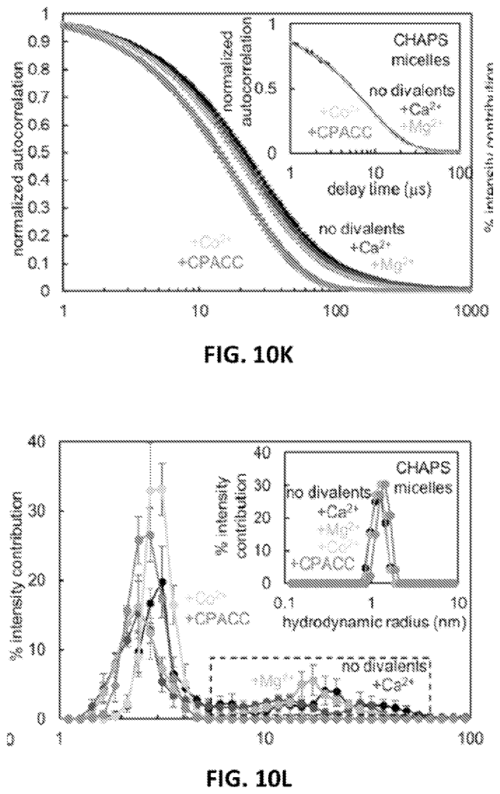

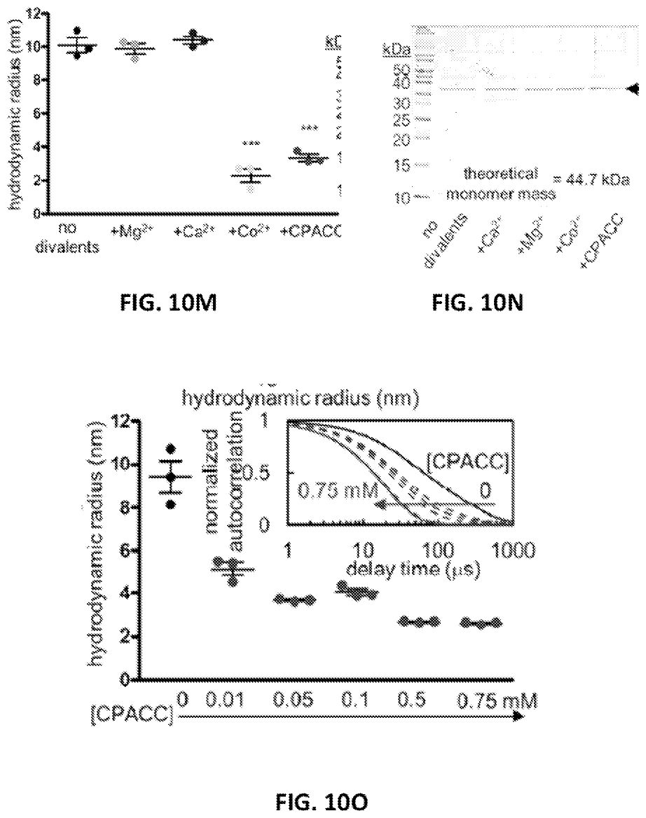

Bacterial CorA blocker selectively inhibits Mrs2 channel activity. Bacterial CorA forms a pentameric complex that selectively drives Mg2+ uptake in an electrogenic manner. CorA is highly selectively inhibited by cobalt and ruthenium derivatives (cobalt(III) hexaammine, ruthenium(III) hexaammine, chloropentaammine cobalt(III) chloride (CPACC; IC₅₀˜100 μM), chloropentaammine ruthenium(III)chloride) with an IC₅₀˜3-100 μM (Kucharski et al., 2000) (FIG. 10A, FIG. 10E). Mrs2 is the eukaryotic homologue of CorA and contains two transmembrane domains with a well-conserved F/Y-G-M-N motif; therefore, CorA blockers were tested to determine if they alter Mrs2-mediated ₘMg²⁺ uptake (Moomaw and Maguire, 2008). Digitonin permeabilized hepatocytes were utilized to measure the inhibitory effect and determine its IC₅₀. Permeabilized cells were treated with CPACC followed by a 1 mM bolus of MgCl₂ (FIG. 10B). A 1 mM Mg²⁺ bolus was added after baseline recording, and the extramitochondrial Mg²⁺ clearance was used as a read-out for Mrs2-mediated ₘMg²⁺ uptake using Mag-Green as a Mg²⁺ indicator. Of all four compounds tested, ruthenium derivatives did not exhibit any inhibitory effect on Mrs2 activity (FIG. 10C). Remarkably, chloropentaammine cobalt(III) chloride(CPACC) exerted complete inhibition of ₘMg²⁺ uptake at 10 μM (FIG. 10D, FIG. 10F). Although we observed limited inhibition by cobalt(III)hexaammine in the permeabilized cell system, its effect in intact hepatocytes were absent (FIG. 10G). CPACC (IC₅₀ for inhibition=0.175 μM) was found to be over 500 times more effective at inhibiting ₘMg²⁺ in permeabilized hepatocytes in comparison with bacterial CorA (FIG. 10D) (Kucharski et al., 2000). We next investigated Mrs2 inhibition in intact hepatocytes loaded with Mag-Green-AM. Mitochondrial Mg²⁺ uptake was measured by confocal microscopy following L-lactate stimulation and was significantly inhibited by 5 μM CPACC (FIG. 10D). Next CPACC was examined to determine if it had any effect on MCU-mediated ₘCa²⁺ uptake in permeabilized cells. Remarkably, CPACC treatment did not inhibit ₘCa²⁺ uptake revealing the specificity of CPACC on Mrs2 (FIG. 10H). The availability of the compound to intact cells was measured using graphite furnace atomic absorption spectroscopy (GFAAS) and bicinchoninic acid in parallel. It is important to note that CPACC accumulates in the cell in a dose-dependent manner (FIG. 10I). Encouraged by the potent inhibitory effect of CPACC and cellular permeability, we next measured the cytotoxicity of CPACC in cells. CPACC was effectively non-toxic in HeLa or HEK293T cells up to 2 mM and 500 μM respectively (FIG. 10J).

Next, CPACC was assessed to determine if it directly affects the assembly of purified human MRS2 using dynamic light scattering (DLS). DLS has been used to determine whether the complex stability of proteins/channels is altered by molecular chaperones or drugs (Kitamura et al., 2006). Because of the high sensitivity of DLS to changes in complex sizes, full-length Mrs2 assembly was evaluated in the presence Ca²⁺ (5 mM), Mg²⁺ (5 mM), Co²⁺ (5 mM) or the Mrs2 blocker CPACC (0.5 mM) (FIG. 10K, FIG. 10L, FIG. 10M, FIG. 10N, FIG. 10O). Remarkably, it was found that Co²⁺ and the Co²⁺ derivative CPACC, but not Mg²⁺ or Ca²⁺, potently decreased the full-length MRS2 complex sizes as determined from the earlier decaying autocorrelation functions, without affecting protein integrity (FIG. 10I-10O). A concentration-response assessment confirmed robust decreases in hydrodynamic radii at all CPACC concentrations tested and as low as 10 μM (FIG. 10O). Together, these data indicate that CPACC distinctly binds to Mrs2 and inhibits the activity.

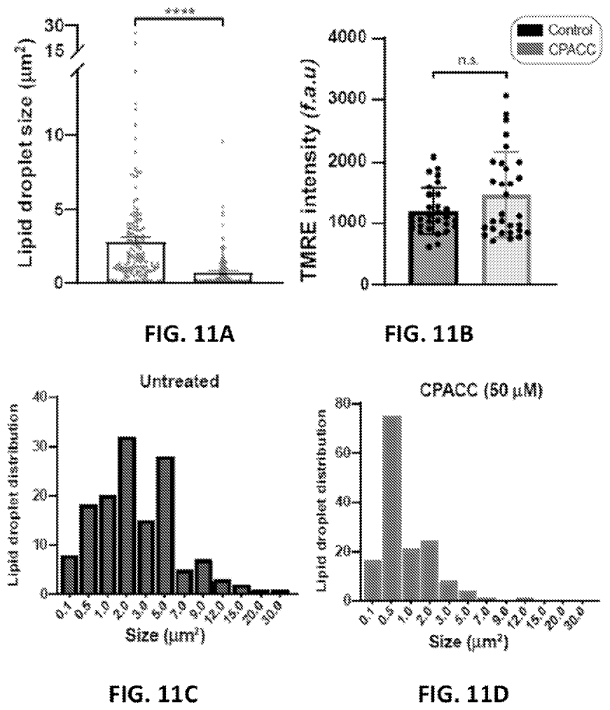

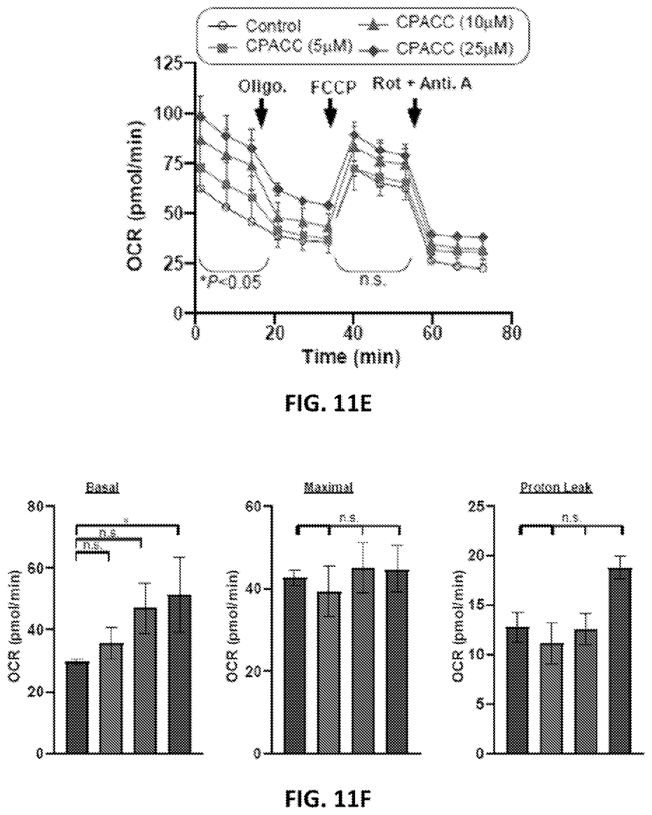

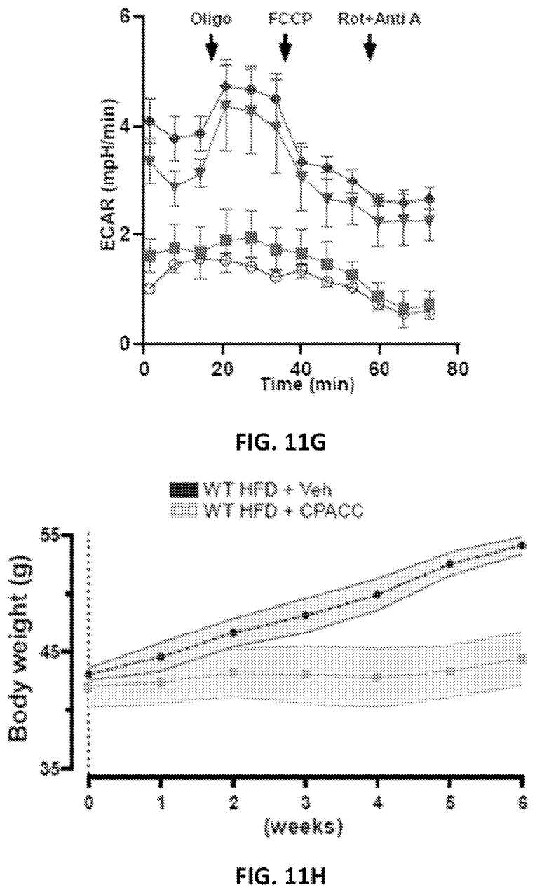

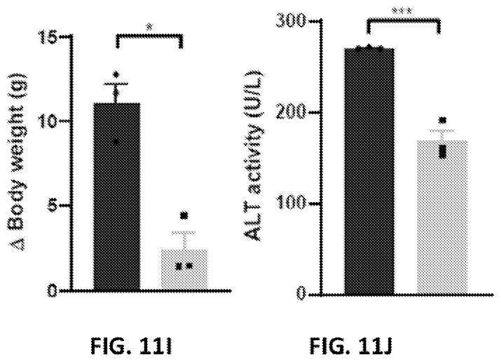

CPACC treatment enhances adipose browning and lowers whole body weight. Given the potent ₘMg²⁺ uptake inhibiting properties and low toxicity of CPACC, we hypothesized that CPACC could mimic Mrs2^{-/-} cellular phenotype. WT hepatocytes isolated from over 12 months old mice were treated with 50 μM CPACC for 48 hours. Control and CPACC treated hepatocytes were stained with BODIPY and TMRE for intracellular tracing using confocal imaging. CPACC treated hepatocytes displayed a marked reduction of lipid droplet size and appeared to dissipate or smear. Importantly, no negative effect on mitochondrial function was observed (FIG. 11A, FIG. 11B, FIG. 11C, FIG. 11D). Next it was determined if CPACC treatment affects the mitochondrial oxygen consumption rate. Freshly isolated WT hepatocytes were treated with 5-25 μM concentrations of CPACC for 16 hours before measurement of oxygen consumption rate. CPACC increased basal mitochondrial respiration without a significant effect on maximal OCR (FIG. 11E, FIG. 11F). Thus, decreased hepatic lipid droplet size by blockade of Mrs2 may result in part from enhanced glucose and fatty acid oxidation, although our data do not preclude the possibility of CPACC-dependent partial stabilization of HIF-1α driving glycolysis and thermogenesis. Since it was observed an enhancement of basal OCR in CPACC treated hepatocytes, the extracellular acidification rate was measured in control and CPACC treated hepatocytes and found it to be higher in the CPACC condition indicating higher glycolytic activity (FIG. 11G). Having observed that pharmacologic blockade of Mrs2 by CPACC reduced lipid droplet size and enhanced mitochondrial function, it was asked whether in vivo administration of CPACC lowers body weight gain. 12 weeks HFD-fed WT mice were treated with CPACC (20 mg/kg) via intraperitoneal injection every three days over 6 weeks. CPACC administration restricted body weight gain (FIG. 11H, FIG. 11I). Plasma ALT levels from CPACC treated mice were lower than those in the control group, suggesting improved liver function (FIG. 11J). Because it was observed browning/beiging in Mrs2^{-/-} iWAT, UCP1 expression by RT-qPCR was tested, confirming induction in the iWAT of CPACC treated mice. Finally, an RT-qPCR analysis was performed which revealed a transcriptional signature of HIF-1α activation in WAT tissue in CPAAC treated HFD mice, suggesting that limiting ₘMg²⁺ acts in part through this pathway to enhance cellular metabolism in multiple tissues.

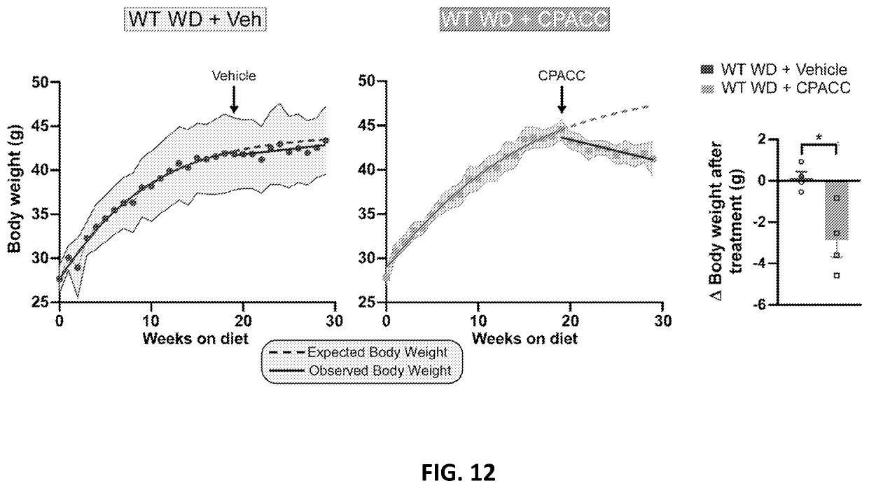

CPACC Treatment Prevents Western Diet Induced Weight Gain and Promotes Weight Loss. 14-week-old WT mice were started on Western diet (n=8) and regularly monitored for 20 weeks and their body weight recorded. At 20 weeks mice started receiving either vehicle or CPACC (40 mg/kg) intraperitoneal (i.p.) injection (n=4 each group) (FIG. 12). The mice were treated for 10 weeks before sacrificing. Utilizing the pretreatment data, the expected body weight was modeled using a Gompertz growth curve. The observed body weight from treatment start to treatment end was modeled using a linear fit. Comparison of the two clearly demonstrates that CPACC not only prevented weight gain but caused weight loss. The reduction in body weight averaged about 2.5 grams, just over a 5% weight loss. By adjusting dosage, administration, and length of treatment, we expect that the weight loss be can even greater based on our collective data. Our data demonstrates key findings to move forward with pharmaceutical testing and initial clinical trials. CPACC is a novel compound that has never been shown to have this phenotype and exposes a completely new druggable target for obesity (Mrs2 in the mitochondria).

### B. Materials and Methods

Cell lines. HEK293 (ATCC #CRL-1573), 293T/17 (ATCC #CRL-11268) and COS-7 (ATCC #CRL-1651) cells were grown in high glucose complete growth medium (high glucose-Dulbecco's modified Eagle's medium (DMEM). HepG2 and HepG2-C3A were grown in normal glucose complete growth medium (Dulbecco's modified Eagle's medium (DMEM) These cell lines were supplemented with 10% (v/v) FBS, 1% (v/v) antimycotic-antibacterial cocktail (Gibco). HK-2 (ATCC CRL2190) cells were maintained in specialized serum-free Keratinocyte-SFM media supplemented with required EGF and BGE (Gibco). All cells kept in a 37° C., 5% CO₂ incubator. Cells lines were detached using Trypsin-EDTA 0.05%, (HEK293, 293T/17, HK-2) or 0.25% (Cos-7, HeLa, HepG2, HepG2-C3A). All transfected cells were grown in corresponding complete growth media supplemented with puromycin (2 μg/ml) or G418 (500 μg/ml).

Animal models. Wild-type (WT) and Mrs2^{-/-} (KO) C57BL/6J mice were housed and maintained in our animal breeding facility with prior approval and accordance with the Institutional Animal Care and Use Committee (IACUC). Mice were fed a Western Diet (WD; Research Diets D09100310), a standard Chow Diet (CD; Envigo 7012), or a High Fat Diet (HFD; Research Diets D12492). For most experiments, WT and KO mice were maintained on the WD or the CD starting at 12 weeks of age. With termination at 12 months. WT mice maintained on a HFD starting at 6 weeks were administered control (saline) or CPACC (20 mg/kg BW in saline) I.P. every 3 days starting after 6 weeks of maintained diet. Body weight, health, and food were measured regularly for all groups. When needed, blood was collected in K₃/EDTA coated tubes and plasma prepared by centrifugation at 1,000×g for 10 min at 4° C.

Isolation and culture of primary murine hepatocytes. Primary adult murine hepatocytes were isolated using portal vein perfusion. After cannulation of the portal vein, the liver is perfused with wash media (35 mM HEPES and 0.75 mM EGTA) followed by digestion media (DMEM, GIBCO, Cat #12320, GIBCO) containing freshly added Collagenase D (380 μg/mL, Worthington) to dissociate extracellular matrix. After perfusion, liver lobes were gently dissected and dissociated in isolation media (DMEM supplemented with 1% (v/v) FBS). The crude hepatocytes were filtered to 100 μm and subjected to three steps of centrifugation-wash cycles (50×g, 4° C., 5 min). After each spin, the pellet was washed in 25 mL of isolation media. The cell's final resuspension is in hepatocyte growth media (Williams E media (Sigma, #W4128) containing 10% (v/v) FBS, 1% (v/v) antibiotic-antimycotic solution, and 200 mM L-glutamine). Finally, cells are counted using trypan-blue exclusion and seeded in hepatocyte growth media according to planned experimental procedures. Hepatocytes are grown in pre-coated collagen culture dishes (Corning BioCoat); or for experiments using confocal microscopy, seeded on in-house collagen coated 25-mm glass coverslips. After 4-8 hours, cell attachment is visually examined, and media is replaced with fresh hepatocyte growth media.

Whole-body respirometry, body composition, and blood chemistry. Mouse metabolic studies were performed at the Penn Diabetes Research Center Rodent Metabolic Phenotyping Core (University of Pennsylvania). A standard 12 h light/dark cycle was maintained throughout the study in a dedicated temperature controlled (20-22° C.) housing room. Mice were acclimated for 5 days before recording. Indirect calorimetry data were recorded using a computer-controlled Promethion Core system (Sable Systems, Las Vegas, NV). Each cage is thermally controlled and equipped with an XYZ beam break array (BXYZ-R, Sable Systems), a voluntary running wheel (WHEEL-M,), and mass measurement modules (2 mg resolution) for food intake and water intake. All animals had ad-libitum access to specified diet and water for 5 days of the study. On the final day, mice were overnight fasted from 1900 to 0900 using a computer-controlled script for automated access to food hopper (Promethion AC-2 Access Control Module) in order to restrict feeding at designated time intervals during the calorimetry run. Respiratory gases are measured with an integrated fuel cell oxygen analyzer, NDIR CO₂ analyzer, capacitive water vapor partial pressure analyzer and barometric pressure analyzer (CGF, Sable Systems). Gas sensors were calibrated using 100% N₂ as the zero reference. Oxygen consumption (VO₂) and carbon dioxide (VCO₂) production are measured for each mouse at 5 min intervals for 20 seconds resulting in a 3 min cycle time. Respiratory Exchange Ratio (RER) is calculated as the ratio of VCO2/VO2. Energy expenditure is calculated using the abbreviated Weir equation: Kcal/hr=60*(0.003941*VO₂+0.001106*VCO₂). Consecutive adjacent infrared beam breaks were counted and converted to distance, with a minimum movement threshold set at 1 cm/s. Voluntary wheel revolutions were also measured continuously as revolutions converted to distance. Data acquisition and instrument control were coordinated by IM-3 software and the obtained raw data were processed using MacroInterpreter v.2.38 (Sable Systems) using an analysis script detailing all aspects of data transformation (One-click macro v.2.45; Sable Systems). EchoMRI nuclear magnetic resonance spectrometer (Echo Medical Systems, Houston, TX) was used to measure whole body lean and fat mass. Blood chemistry parameters were determined as followed: plasma cholesterol with an enzymatic assay (Stanbio), glucose using a ReliON glucometer, an insulin using ELISA.

Histology and quantification. Tissues were fixed in 10% neutral buffered formalin and washed in 70% ethanol overnight before paraffin embedment and generation of unstained slides. Paraffin embedment, mounting, slide preparation, and standard staining was done by the Histology Laboratory in UT Health San Antonio Department of Pathology & Laboratory Medicine with detailed protocols on file. Briefly, tissues are dried, paraffin embedded, sectioned, and placed on slides. For H&E and Masson's Trichome staining, sections were processed according to the Histology Laboratory's established protocols. Unstained liver tissue sections were used to conduct IHC (immunohistochemistry) in-house. Sections were deparaffinized then rehydrated, followed by antigen retrieval (0.01 M citrate buffer). Endogenous peroxidase blocking was done using 3% H₂O₂ for 10 minutes. Avidin and biotin blocking was conducted using Vector Laboratories Avidin/Biotin Blocking Kit (SP02001). Nonspecific binding was blocked by 1 hour incubation in normal horse serum (NHS, Vector Laboratories, S-2000-20) containing 0.1% Tween-20 in 0.01M PBS and 10% BSA. Unconjugated goat anti-mouse CD-1/PECAM-1 (14 μg/mL; R&D Systems, AF3628) primary was applied overnight at 4° C. in 10% NHS. Sections were incubated with biotinylated ready-to-use horse anti-goat IgG secondary (Vector Laboratories, BP-9500-50) for 1 hour at room temperature. Secondary development was done using the VECTASTAIN Elite ABC-HRP kit (Vector Laboratories PK-6100) and DAB reagent according to manufacturer's recommendations. Developed sections were counterstained with Mayer's Hematoxylin. Thorough washing with PBS was conducted after each step. Slides were imaged using a Olympus light microscope and imaged at 20× magnification. Vascular density was quantified from H&E-stained slides. A digital filter was placed over selected images for vascular clarity and the number of micro vessels observed in each image was recorded. 2-3 slides (different mice) per group were used.

Plasma alanine transaminase activity and citrate measurements. Liver function and damage was determined by measuring alanine transaminase (ALT). The ALT activity in the plasma was determined using the Alanine Transaminase Activity Assay Kit (Abcam ab105134) following manufacturer's protocol. The plasma ALT levels over two different time points were obtained and corresponding ALT activity was determined by reading in a plate reader at 570 nm. The generation of citrate in the TCA cycle and concomitant extrusion from the liver into the blood stream was investigated by estimating the levels of citrate in the plasma. Citrate levels were determined using the Citrate Assay Kit (Abcam ab83396) by reading in a plate reader at 570 nm following the manufacturer's protocol. When required, deproteination was accomplished using a trichloroacetic acid precipitation kit (Abcam ab204708) for tissue and cells, or 10,000 MWCO spin column for plasma.

RNAseq and analysis. RNAseq data generation was contracted with NovoGene. Libraries were constructed using poly-T magnetic beads following by fragmentation, cDNA synthesis, adaptor ligation, and finally PCR. Quality control was conducted by removing low quality reads or those containing the adaptor or poly-N strings. Reads were aligned to the reference genome using Hisat2 v2.0.5. FeatureCounts v1.5.0-p3 was used to count mapped reads and FPKM values were subsequently calculated to correct for gene length and sequencing depth. Consistency between biological replicates was confirmed by correlation analysis. DESeq2 v1.20.0 was used for differential expression analysis; P-values were adjusted using the Benjamini-Hochberg method and statistical significance was considered by an adjusted P-value<0.05. Gene set enrichment analysis (GSEA) was done using the publicly available GSEA analysis tool from Broad Institute (URL www.broadinstitute.org/gsea/index.jsp). The R package ClusterProfile was used to test for significant enrichment of KEGG pathways. Following NovoGene's preliminary analysis, further in-house analysis was conducted. Normalized FPKM values for heatmaps were generated using the STANDARDIZE formula in Microsoft Excel and each gene was normalized individually. Relative mRNA abundance was calculated by normalization from the mean FPKM for the gene and genes were normalized independently. Finally, the Sankey plot was generated using the SankeyMATIC tool (URL sankeymatic.com/).

Immunoblotting. Crushed tissues were homogenized in lysis buffer (ThermoFisher) on ice for one minute (20-30 strokes) using an automatic Dounce homogenizer set to 2000 RPM. Whole cell lysate was prepared in lysis buffer (Abcam) using 3 sets of 3-second sonication (power level 3) intervals on ice. Protein concentration was estimated using Pierce BCA assay kit and samples were prepared and heated for 90° C. for 5 minutes. Equal amounts of protein were loaded and separated on 4%-12% Bis-Tris polyacrylamide gel (Thermo Fisher Scientific), transferred to a PVDF membrane, blocked for 1-2 h using 5% fat free skim milk, washed and finally, probed with corresponding antibodies as specified below. Antibodies were from Cell Signaling Technology (HIF-1α and Hydroxy HIF-1α dilution 1:3000, MCU dilution 1.5000, MICU1 dilution 1:3000), Abcam Abcam (CPT-1a, CPT-2, OXPHOS cocktail dilution 1:3000), ZYMED Laboratories (β-actin; dilution 1:1,000), and Amersham (secondary antibodies conjugated with peroxidase). Development was done using X-ray film using a series of timed exposures and Image J was used for densitometric analysis on scanned film images.

Mitochondrial oxygen consumption rate. Primary murine hepatocytes were plated on in-house collagen coated 96-well Seahorse XF Cell Culture Microplates (Agilent) at a density of 4×10⁵ cells/well. Cells were maintained in their normal growth media until 1 hour before assay start time. Hepatocytes were treated with 5, 10 and 25 μM of chloropentammine cobalt(III) chloride (CPACC) for 1 hour at 37° C. Media was changed to Seahorse XF Cell Mito Stress Test Kit (Agilent) assay media supplemented with glucose, glutamine, pyruvate, and HEPESn with concentrations equivalent to that of the growth media 1 hour before the experiment start time. After media change, per manufacturer instructions, cells were placed in a CO₂-free incubator for 1 hour. Oxygen consumption rate (OCR) was measured at 37° C. in an XF96 extracellular flux analyzer (Seahorse Bioscience, Agilent) calibrated using Seahorse XF Calibrant solution (Seahorse Bioscience, Agilent) in a CO₂-free incubator overnight. Respiratory chain inhibitors (2 μM oligomycin, 5 M FCCP, and a mixture of 1 μM antimycin A and 1 μM rotenone) were added at the indicated time points. Data was collected using Agilent Seahorse Wave 2.6.1 Desktop software and analyzed using GraphPad Prism version 8 (Irrinki et al., 2011; Tomar et al., 2016).

Confocal microscopic examination of cellular lipid localization and maintenance. Primary murine hepatocytes were stained with the lipid indicator BODIPY (ex/em 493/503 nm, 1 μg/ml) and mitochondrial membrane potential indicator tetramethylrhodamine, ethyl ester (TMRE, ex/em 556/610 nm, 100 nM) in serum-free conditions for 30 min (5% CO₂) and washed before imaging. (Tomar et al., 2019). For CPACC-treated experiments, hepatocytes were acquired from 12 month-old mice and cells were treated with CPACC (50 μM) for 12 hours. All confocal microscopic images were acquired using a Leica SP8 confocal microscope (Manheim, Germany) coupled with a temperature-controlled environmental chamber. Leica Application Suite X was used to quantify lipid-mitochondrial colocalization using line scan analysis followed by smooth curve fitting. ImageJ was used to calculate Pearson's and Mander's colocalization coefficients. The lipid droplet sizes and mitochondrial membrane potential were quantified using both Leica Application Suite X and ImageJ. All data was analyzed using GraphPad Prism v8.

Evaluation of dose dependent Mrs2 inhibition by confocal live cell imaging system. Primary murine hepatocytes were treated with varying doses (1, 2, 5, 10, 25 μM) of CPACC, or control, for 12 hours. To visualize Mg²⁺ and the mitochondria, cells were stained with 2.5 mM Magnesium Green-AM (ex/em 488/510 nm) and 1 mM MitoTracker Deep Red FM (ex/em 644/665 nm) for 30 minutes in their normal growth conditions. Using the Leica SP8 confocal microscope, time lapse images were collected every 3 seconds. After 30 seconds of baseline recording, the cells were stimulated with lactate (5 mM). Data was quantified using Leica Application Suite X.

Visualization of citrate flux in live cells using confocal microcopy. Primary murine hepatocytes were grown and seeded as previously described. HepG2 cells were grown on 0.1% gelatin coated glass coverslips. After overnight growth, cells were transiently transfected with 2 μg of genetically encoded biosensors, CMV-Citron or CMV mito-Citron1 (deposited by Robert Campbell) at Addgene, #134303 and #134305; GFP ex/em 488/510 nm) using Lipofectamine 3000 transfection reagent. After 24 hr, transfected hepatocytes were infected with adenoviruses (Vector BioLabs), Ad-RFP and Ad-Mrs2(mut)mRFP-FLAG (MOI 10). After 48 h of infection, the cells were washed and imaged using the Leica SP8 Confocal microscope under 60×oil immersion. After 30 seconds of baseline recording, cells were stimulated with 20 mM of glucose (hepatocytes) or 16.7 mM (HepG2) and the corresponding fluorescence emissions in the cytosol and mitochondria were recorded. The citrate transient-generated fluorescence emission was quantified using Leica Application Suite X and analyzed using GraphPad Prism v8.

Spectrofluorimetric measurement of mitochondrial Mg²⁺ and Ca²⁺ dynamics and citrate mediated MagGreen fluorescence quenching. Fluorescence measurements were conducted in a multiwavelength excitation dual wavelength emission spectrofluorometer (Delta RAM, PTI, HORIBA). Cells were washed with Ca²⁺ and Mg²⁺ free DPBS, pH 7.4. Following centrifugation (50×g, 4° C., 5 min), approximately 4-5×106 cells were resuspended and permeabilized using 40 μg/mL digitonin in 1.5 mL of intracellular medium (ICM) (mM, 120 KCl, 10 NaCl, 1 KH₂PO₄, 20 HEPES-Tris, pH 7). Suspension was additionally supplemented with succinate (5 mM), ATP, and a fluorescent dye. Magnesium measurements were performed using K⁺/ATP (1.5 mM) and Mag Green (0.5 mM); meanwhile, calcium measurements were performed using Mg²⁺/ATP (1.5 mM) and Fura-2FF (1 μM). Mag-Green has an excitation of 505 nm and emissions of 535 nm and 595 nm based on Mg²⁺ binding. Fura2-FF has excitations 340 nm and 380 nm based on Ca²⁺ binding and emits at 510 nm). Changes in extramitochondrial Mg²⁺ ([Mg²⁺]ₒᵤₜ) or Ca²⁺ ([Ca²⁺]ₒᵤₜ) was used as an indicator of mitochondrial uptake of these ions. After a 400 sec background acquisition period, cells were pulsed with either a single bolus of 1 mM Mg²⁺ or multiple 20 μM Ca²⁺ pulses followed by the mitochondrial uncoupler, FCCP (2 μM). To measure the inhibition of ₘMg²⁺ uptake, permeabilized hepatocytes in ICM were supplemented with different concentrations (0.05, 0.1, 0.5, 1, 2, 5, 10, 25 μM) of CPACC, or control, and pulsed with a bolus of 1 mM Mg²⁺ at 450 s followed by FCCP (2 μM) at 1000 s. This set of experiments was also conducted using hexaammine Co(III) chloride at the same dosages; additionally, ruthenium ion based inhibition of ₘMg²⁺ was investigated using hexaammine Ru(III) chloride (5 μm) and chloropentammine Ru(III) chloride (5 μM). The ₘMg²⁺ uptake rate was calculated from the linear portion of the traces immediately after Mg²⁺ addition. Since citrate acts as an endogenous chelator of magnesium ions, this property can be exploited to visualize citrate production as an inverse function of MagGreen (Mg²⁺ fluorescent dye) intensity. Hepatocytes were pulsed with 1 mM Mg²⁺ after a 600 s baseline recording period followed by 5 mM oxaloacetate (OAA) at 800 s. All experiments were done at 37° C. with constant stirring.

Cellular uptake assay. Hepatocytes were treated with varying doses of CPACC for 30 minutes, cells were washed in Ca²⁺ and Mg²⁺ free DPBS, and cell pellets flash-frozen. Pellets were suspended in ice cold lysis buffer (0.1% Triton X-100 and 0.2% HNO₃ in ultrapure water). The suspension was vortexed for 30 s and incubated on ice for 45 min. The cell lysate was centrifuged, and the supernatant was transferred to a clean tube prior to analysis. The Co³⁺ content of the lysate was determined using graphite furnace atomic absorption spectroscopy (GFAAS) and was normalized to the protein content of the sample, which was determined using the bicinchoninic acid (BCA) assay kit following manufacturer instructions (ThermoFisher). Results are reported as the average mass ratio of Co to protein (pg/μg) in each sample±SEM.

MRS2 cloning, expression, and purification. The full length human MRS2 (NCBI accession NP_065713.1), identified as MRS258-443 (i.e. residues 58-443) and excluding the mitochondrial targeting sequence, was cloned into pET-28a using Ndel and Xhol restriction sites. The sequence and frame of the MRS258-443 coding insert within the pET-28a vector was confirmed by Sanger DNA sequencing. Transformed BL21 (DE3) codon+Escherichia coli were grown in Luria-Bertani (LB) broth supplemented with 60 mg/mL kanamycin to an optical density (600 nm) of≈0.6-0.8 at 37° C. Expression was induced upon addition 300 mM isopropyl β-D-1-thiogalactopyranoside (IPTG), and growth was continued overnight. The protein was purified from the harvested cells under native conditions according to the HisPur nickel-nitriloacetic acid (Ni-NTA) manufacturer guidelines (ThermoFisher). After elution from the Ni-NTA beads using buffer containing 20 mM Tris, 150 mM NaCl, 1 mM DTT, 10 mM CHAPS, 325 mM imidazole, pH 8.0, the 6×His-MRS258-443 was dialyzed in the same buffer without imidazole and thrombin digested (≈1 U/mg protein) to remove the 6×His tag. A final size exclusion chromatography (SEC) purification step was performed through a Superdex 200 10/300 GL column connected to an AKTA Pure FPLC (GE Healthcare). The buffer for the SEC and all experiments was 20 mM Tris, 150 mM NaCl, 1 mM DTT, 10 mM CHAPS, pH 8.0.

Dynamic light scattering (DIS). A Dynapro Nanostar (Wyatt) equilibrated to 20° C. was used for all DLS experiments. Protein samples ≈0.5 mg/mL in 20 mM Tris, 150 mM NaCl, 1 mM DTT, 10 mM CHAPS, pH 8.0 without or with 5 mM CaCl₂, 5 mM MgCl₂, 5 mM CoCl₂ or 0.5 mM CPACC were centrifuged at 12,000×g for 5 min before a 5 mL aliquot of the supernatant was removed and loaded into a JC501 cuvette (Wyatt). The sample was equilibrated for 5 min at 20° C. before 10 autocorrelation function acquisitions of 5 s each were recorded and averaged. Two aliquots (technical replicates) from each sample were averaged, and each experimental condition was performed on three independent/individual protein expression preparations (biological replicates). Autocorrelation functions were deconvoluted using the regularization algorithm to extract polydisperse distributions of hydrodymic radii and cumulants algorithm to extract weight-averaged monodisperse hydrodynamic radii, using the accompanying Dynamics (v7.8.1.3) software (Wyatt). The dependence of hydrodynamic size on CPACC concentration was determined using ≈0.5 mg/mL protein supplemented with 0.01 mM, 0.05 mM, 0.1 mM, 0.5 mM or 0.75 mM CPACC, using a similar experimental setup as described above and cumulants analysis.

Statistical Analysis. Two-tailed Student's t test and One Way ANOVA with Tukey's multiple comparisons were used as indicated. GraphPad Prism version 8 was used for statistical testing and regression analysis. Data is represented as mean+/−SEM unless otherwise indicated.

1. A method of ameliorating or preventing a metabolic syndrome comprising administering an effective amount of chloropentaammine cobalt(III) chloride (CPACC) or a variant thereof to a subject.

2. The method of claim 1, wherein CPACC or a derivative thereof is administered at a dose of between 100 and 500 μg or mg.

3. The method of claim 1, wherein CPACC or a derivative thereof is formulated as a tablet, a capsule, a concentrate, a powder, a drink, a baked good, chocolate, caramel, and/or snack.

4. The method of claim 1, wherein the CPACC derivative has a chemical structure of Formula II. 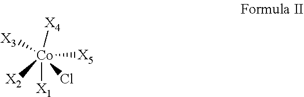
wherein X₁, X₂, X₃, X₄, and X₅ are independently selected from a halogen, amine, nitro, mercapto, hydroxyl, C1 to C4 alky, Cl to C4 heteroalkyl, alkoxy, alkylthio, or alkylamino.

5. The method of claim 4, wherein X₁, X₂, X₃, X₄, and X₅ are independently chlorine (Cl), bromine (Br), fluorine (F), or iodine (I).

6. The method of claim 4, wherein 1, 2, 3, 4, or 5 of X₁, X₂, X₃, X₄, and X₅ are chlorine.

7. The method of claim 1, wherein CPACC or a derivative thereof is administered orally.

8. The method of claim 1, wherein CPACC or a derivative thereof is administered once a day.

9. A method of ameliorating diet-induced metabolic syndrome comprising administering chloropentaammine cobalt(III) chloride (CPACC) or a variant thereof to a subject.

10. A method for treating obesity, cardiovascular diseases (CVD) complications, diet-induced hepatocellular carcinomas (HCC), breast cancer, ischemic injury comprising administering an effective amount of CPACC or a derivative thereof to an obese subject, subject with cardiac disease, and/or a subject with hepatocellular carcinoma (HCC).

11. The method of claim 10, wherein the subject has a body mass index (BMI) of 30 or greater.

12. The method of claim 10, wherein the subject is diagnosed with pre-diabetes.

13. A method for treating pre-diabetes comprising administering an effective amount of CPACC or a derivative thereof to a pre-diabetic subject.

14. A method for treating diabetes comprising administering an effective amount of CPACC or a derivative thereof to a subject having diabetes.

15. A composition comprising CPACC or a derivative thereof, the CPACC derivative having a chemical structure of Formula II 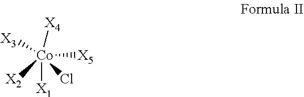
wherein X₁, X₂, X₃, X₄, and X₅ are independently selected from a halogen, amine, nitro, mercapto, hydroxyl, C1 to C4 alky, C1 to C4 heteroalkyl, alkoxy, alkylthio, or alkylamino.
